SEQUENCE LISTING (19 sequences)

SEQ ID NO: 1 - protein, 290 aa, Homo sapiens
MANCEFSPVSGDKPCCRLSRRAQLCLGVSILVLILVVVLAVVVPRWRQQWSGPGTTKRFPTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 452 aa, Artificial Sequence
  daratumumab heavy chain
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, Artificial Sequence
  daratumumab light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 122 aa, Artificial Sequence [sequence omitted]

SEQ ID NO: 5 - protein, 107 aa, Artificial Sequence [sequence omitted]

SEQ ID NO: 6 - protein, 5 aa, Artificial Sequence
  daratumumab HCDR1
SFAMS

SEQ ID NO: 7 - protein, 17 aa, Artificial Sequence
  daratumumab HCDR2
AISGSGGGTYYADSVKG

SEQ ID NO: 8 - protein, 13 aa, Artificial Sequence
  daratumumab HCDR3
DKILWFGEPVFDY

SEQ ID NO: 9 - protein, 11 aa, Artificial Sequence
  daratumumab LCDR1
RASQSVSSYLA

SEQ ID NO: 10 - protein, 7 aa, Artificial Sequence
  daratumumab LCDR2
DASNRAT

SEQ ID NO: 11 - protein, 10 aa, Artificial Sequence
  daratumumab LCDR3
QQRSNWPPTF

SEQ ID NO: 12 - protein, 120 aa, Artificial Sequence [sequence omitted]

SEQ ID NO: 13 - protein, 109 aa, Artificial Sequence
  isatuximab variable light region
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRT

SEQ ID NO: 14 - protein, 5 aa, Artificial Sequence
  isatuximab HCDR1
DYWMQ

SEQ ID NO: 15 - protein, 17 aa, Artificial Sequence
  isatuximab HCDR2
TIYPGDGDTGYAQKFQG

SEQ ID NO: 16 - protein, 11 aa, Artificial Sequence
  isatuximab HCDR3
GDYYGSNSLDY

SEQ ID NO: 17 - protein, 11 aa, Artificial Sequence
  isatuximab LCDR1
KASQDVSTVVA

SEQ ID NO: 18 - protein, 7 aa, Artificial Sequence
  isatuximab LCDR2
SASYRYI

SEQ ID NO: 19 - protein, 9 aa, Artificial Sequence
  isatuximab LCDR3
QQHYSPPYT

(19) United States
(12) Patent Application Publication
(10) Pub. No.: US 2022/0135695 A1
(43) Pub. Date: May 5, 2022

(54) ANTI-CD38 AGENTS FOR DESENSITIZATION AND TREATMENT OF ANTIBODY-MEDIATED REJECTION OF ORGAN TRANSPLANTS

(71) Applicant: Cedars-Sinai Medical Center, Los Angeles, CA (US)

(72) Inventors: Stanley C. JORDAN, Manhattan Beach, CA (US); Noriko AMMERMAN, Los Angeles, CA (US); Ashley VO, Porter Ranch, CA (US); Mieko TOYODA, Irvine, CA (US); Robert VESCIO, Los Angeles, CA (US)

(73) Assignee: Cedars-Sinai Medical Center, Los Angeles, CA (US)

(21) Appl. No.: 17/435,159

(22) PCT Filed: Mar. 9, 2020
(86) PCT No.: PCT/US20/21690

§ 371 Date: Aug. 31, 2021

Related U.S. Application Data
(60) Provisional application No. 62/815,958, filed on Mar. 8, 2019.

(51) Int. Cl. C07K 16/28; A61K 31/436; A61K 31/365; A61K 39/395; A61P 37/06

(52) U.S. Cl. CPC C07K 16/2896; A61K 31/436; A61K 2039/545; A61K 39/3955; A61P 37/06; A61K 31/365

(57) ABSTRACT

Methods and systems for desensitizing a human leukocyte antigen (HLA) sensitized subject to prepare for an organ transplant with an improved transplant survival and function, and/or treating or reducing the likelihood of antibody mediated rejection (ABMR) of an organ transplant in a subject are provided, generally including administering an effective amount of an anti-CD38 antibody or a CD38-targeting therapy to reduce the symptoms or ABMR or HLA levels. The subject in the methods may have developed or is experience drug-resistant sensitization, and to whom standard techniques like intravenous immunoglobulin and plasmapheresis are ineffective.

### CROSS-REFERENCE TO RELATED APPLICATIONS

This application includes a claim of priority under 35 U.S.C. § 119(e) to U.S. provisional patent application No. 62/815,958, filed Mar. 8, 2019, the entirety of which is hereby incorporated by reference.

### FIELD OF INVENTION

This invention relates to anti-CD38 antibodies and CD38-targeting therapies in prophylaxis and therapeutic treatment of patients with anti-human leukocyte antigen antibodies or experiencing standard-of-care resistant antibody-mediated rejection.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Alloimmune responses are responsible for the majority of renal allograft failures which total about 5,000 per year in the U.S. Antibody-Mediated Rejection (ABMR) is a severe form of rejection mediated by B-cells, plasma cells and antibodies. The consequences to the patients with ABMR are often severe with high rates of graft loss and poor patient survival. Patients returning to the transplant list after allograft failure now represent the fourth largest category for new patient listings in the U.S. These patients represent a major problem for transplant centers as they are highly-human leukocyte antigen (HLA) sensitized and unlikely to receive another transplant without significant desensitization. Today, this represents one of the most important and potentially achievable goals of transplant medicine. There are currently no FDA approved drugs in this category.

Antibodies to HLA antigens have a strong impact on mediation of allograft injury and loss and remain a persistent and often impenetrable barrier to successful transplantation for thousands of patients on renal transplant lists worldwide. Pre-formed or de novo donor specific antibodies (DSAs) activate complement, induce endothelial cell proliferation and mediate antibody dependent cytotoxicity (ADCC), which leaves the recipient highly HLA sensitized, suffering from persistent immune attack on the allograft, and results in a progression of interstitial fibrosis, tubular atrophy (IF/TA), and allograft dysfunction and loss. Patients returning to dialysis have little hope of receiving a subsequent transplant and often face a higher risk of death on dialysis. DSAs are also known to accelerate atherosclerosis in the allograft thus hastening the vascular demise of the kidney.

Alloantibodies are a major deterrent to access to and success of life-saving organ transplants. Despite advancements in desensitization, designing efficient and effective means of removal of pathogenic HLA antibodies remains a significant medical challenge. There are a number of notable deficiencies of the existing desensitization protocols. For example, failure of current therapies to substantially completely remove DSAs before transplantation results in the risk for early antibody-mediated rejection. There is also a risk of rebound DSA formation post-transplant with attendant injury to the allograft, both acute and chronic. And some of current protocols, especially those utilizing complement inhibitors to prevent chronic antibody mediated rejection (cABMR), still fail to deliver desirable outcomes.

To increase renal transplant rates in sensitized patients, new protocols for HLA desensitization have emerged. These approaches require the application of intravenous immunoglobulin (IVIG), rituximab and plasma exchange (plasmapheresis, PLEX). There is a growing interest in developing new immune-modulatory drugs that are less expensive and more convenient for improving antibody reduction in transplantation. However, instances have occurred where patients' response to these protocols are ineffective. IVIG with PLEX does not always prove dependable.

As such, an unmet medical need exists to improve the renal or other organ's transplant survival by reducing or eliminating pre-existing anti-HLA antibodies to a level that would allow patients to receive life-saving organ transplants or by reducing or eliminating donor specific HLA antibodies and treating antibody-mediated rejection to improve the transplant function and survival.

Therefore, it is an object of the present application to provide a method for desensitization of patient with anti-human leukocyte antigen antibodies.

It is also an object of the present application to provide methods and systems to treat patients experiencing standard-of-care resistant antibody-mediated rejection of organ transplants.

### SUMMARY OF THE INVENTION

The following embodiments and aspects thereof are described and illustrated in conjunction with compositions and methods which are meant to be exemplary and illustrative, not limiting in scope.

Provided are methods of reducing or removing donor specific anti-human leukocyte antigen (HLA) antibodies in a HLA-sensitized subject, and/or treating or reducing the severity of antibody-mediated rejection of the solid organ transplant in a patient with a high level of HLA antibodies, which can improve survival and function of a solid organ transplant, especially in those having established and drug-resistant antibody-mediated rejection or to whom standard-of-care treatment has failed. The methods can include administering to the patient an effective amount of an anti-CD38 antibody, a CD38-binding fragment of an antibody, immune cells expressing a chimeric antigen receptor (CAR) that comprises at least a CD38-targeting region, a polynucleotide encoding the CAR, a vector comprising the polynucleotide, or a combination thereof. In some aspects, the methods can further include selecting a patient experiencing or having experienced antibody-mediated rejection of an organ transplant. In other aspects, the methods can further include selecting a patient with donor specific anti-HLA antibodies in the serum. In various aspects, the anti-CD38 antibody or CD38-binding fragment thereof can be selected from the group consisting of daratumumab, isatuximab, MOR-202, GBR-1342, AMG-424, TAK-169, MT-4019ND, STI-6129, A-145D, EDC-8, or a combination thereof.

In some embodiments of the methods, the anti-CD38 antibody or an antigen-binding fragment thereof can contain a variable heavy region (V_{H}) of amino acid sequence of SEQ ID No: 4.

In some embodiments of the methods, the anti-CD38 antibody or an antigen-binding fragment thereof can contain a variable light region (V_{L}) of amino acid sequence of SEQ ID No: 5.

In some embodiments of the methods, the anti-CD38 antibody or an antigen-binding fragment thereof can contain a variable heavy region (V_{H}) of amino acid sequence of SEQ ID No: 4 and a variable light region (V_{L}) of amino acid sequence of SEQ ID No:5.

In some embodiments of the methods, the anti-CD38 antibody or an antigen-binding fragment thereof can contain a variable heavy chain polypeptide including heavy chain complementarity determining regions (HCDR) 1 (HCDR1), 2 (HCDR2) and 3 (HCDR3) sequences of SEQ ID NOs: 6, 7 and 8, respectively, and a variable light chain polypeptide including light chain complementarity determining regions (LCDR) 1 (LCDR1), 2 (LCDR2) and 3 (LCDR3) sequences of SEQ ID NOs: 9, 10 and 11, respectively.

In some embodiments of the methods, the anti-CD38 antibody can be daratumumab.

In some embodiments of the methods, the anti-CD38 antibody or CD38-binding fragment of an antibody can be administered at an amount equivalent to 10-20 mg intravenously/kg of the subject/week for at least four weeks.

In further embodiments of the methods, the subject can be one who has undergone standard-of-care treatment comprising one or more of immunoglobulin administration (IVIG), rituximab administration and plasma exchange (PLEX), and the subject's response to the standard-of-care treatment is ineffective. In yet further embodiment of the methods, the subject can be one who is further resistant or has acquired resistance to immunosuppressive treatment with one or more of eculizumab, thymoglobulin, bortezomib, carfilzomib, basiliximab, mycophenolate mofetil, tacrolimus and corticosteroids.

Further embodiments provide a method of (1) treating or reducing the severity of antibody-mediated rejection of a solid organ transplant and/or (2) desensitizing a subject characterized by reduced level of HLA antibodies, compared to a value obtained from the same subject prior to the desensitization step, comprising administering to the subject in need thereof an anti-CD38 antibody comprising heavy chain complementarity determining regions (HCDR) 1 (HCDR1), 2 (HCDR2) and 3 (HCDR3) sequences of SEQ ID NOs: 6, 7 and 8, respectively, and light chain complementarity determining regions (LCDR) 1 (LCDR1), 2 (LCDR2) and 3 (LCDR3) sequences of SEQ ID NOs: 9, 10 and 11, respectively.

Various aspects of the disclosed methods can include reduced presence of HLA antibodies in the patients following administration of daratumumab, or a CD38-binding fragment thereof, compared to a value obtained from the same patient prior to the administration. The anti-CD38 antibody can be administered before organ transplantation in the patient. The anti-CD38 antibody can be administered after organ transplantation in the patient.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A is a bar graph showing results from LUMINEX assay for HLA class I & II antibodies (with respective antigen listed below each bar), expressed as mean fluorescent intensity (MFI), in a heart transplant candidate in Example 2 after desensitization treatment with daratumumab. This patient showed a significant reduction in both class I and class II antibodies post-daratumumab (71% reduction in mean MFI values). Patient had received multiple previous desensitization therapies without impact on HLA class I & class II MFI values.

FIG. 1B depicts flow cytometry results of peripheral B-cell and T-follicular (Tfh) subsets of the patient in Example 2 before and after desensitization with daratumumab, compared to a normal control subject. Compared to normal control, the patient showed more B-reg, plasma cells, plasmablast and Tfh cells prior to daratumumab treatment. After daratumumab treatment, B-regs, plasma cells and plasmablast were eliminated and Tfh cells reduced. This coincided with significant reductions in HLA class I & II antibodies as shown in FIG. 1A.

FIG. 2A is a bar graph showing results from LUMINEX assay for HLA class I &II antibodies (with respective antigen listed below each bar), expressed as mean fluorescent intensity (MFI), in the patient of Example 1, pre- and post-daratumumab therapy. Disparate results were seen for HLA class I compared to class II. A significant and persistent reduction of HLA class I antibodies was seen that did not rebound over a 4M observation period post-daratumumab. However, there was no impact on HLA class II antibodies including the DSA to DQ5. There was also rebound in several class II antibodies and appearance of de novo HLA class II antibodies.

FIG. 2B depicts flow cytometry results of CD4+ T-cells pre- and post-daratumumab therapy of the patient in Example 1. There is an increase in peripheral CD4+ T-cells after daratumumab therapy that coincides with worsening of CMR (Banff 1B) seen on the post-daratumumab biopsy. B-regs, plasma cells and plasmablast showed complete elimination post-daratumumab therapy.

FIG. 3A shows microscopic images of renal biopsy of the patient in Example 2 before the daratumumab treatment. First row on the left is from the patient's allograft biopsy performed in January of 2018, which revealed acute cell mediated rejection, Banff '17 grade 1A, with moderate tubulitis (7 leukocytes per 10 tubular epithelial cells), demonstrated in the tubule in the center of the image (Periodic acid Schiff, magnification 600×). First row in the middle and on the right shows a subsequent biopsy performed one month later in, which revealed worsening acute cell mediated tubulointerstitial and vascular rejection with extensive interstitial inflammation (first row in the middle) Period acid Schiff 200×, severe tubulitis (indicated by red arrows in the first row, on the right) Jones methenamine silver 600×, and focal endarteritis (second row on the left), Banff '17 Grade 2A, Periodic acid Schiff 400×. In addition, the biopsy also showed features consistent with C4d negative acute antibody mediated rejection with severe peritubular capillaritis (second row in the middle), Jones methenamine silver 200×, and focal glomerulitis (second row on the right), Periodic acid Schiff 400×.

FIG. 3B shows microscopic images of renal biopsy of the patient in Example 2 after the daratumumab treatment. The biopsy revealed an extensive, diffuse interstitial inflammatory infiltrate which involved areas of moderate parenchymal scarring (i-IF/TA, on the left) Trichrome stain 100×, with multifocal, severe tubulitis (in the middle) Periodic acid Schiff 200×. The findings were consistent with chronic active cell mediated rejection, Banff '17 Grade 1B. While there was residual mild peritubular capillaritis, it was less severe than the previous biopsy (on the right), unremarkable glomerulus without glomerulitis. These findings did not meet diagnostic criteria for acute antibody mediated rejection.

FIG. 4 depicts the levels of total mean fluorescence intensity (MFI) of class I DSAs and class II DSAs, each before (denoted “Pre Daratumumab”) and after (denoted “Post Daratumumab”) Daratumumab treatment of the patient (N=1) in the Example.

FIG. 5 depicts the levels of average mean fluorescence intensity (MFI) of class I DSAs and class II DSAs, each before (denoted “Pre Daratumumab”) and after (denoted “Post Daratumumab”) Daratumumab treatment of the patient (N=1) in the Example.

FIG. 6 depicts the Banff scores of the patient (in the Example) before (denoted “Pre Daratumumab”) and after (denoted “Pre Daratumumab”) Daratumumab treatment. Denotations, Banff lesion score “g” (Glomerulitis) and Banff lesion score “ptc” (Peritubular Capillaritis), indicate microvascular inflammation. Banff lesion score “cg” is based on the presence and extent of glomerular basement membrane (GBM) double contours or multilamination in the most severely affected glomerulus. Banff lesion score “v” evaluates the presence and the degree of inflammation within the arterial intima. Banff lesion score “C4d” evaluates the extent of staining for C4d on endothelial cells of peritubular capillaries and medullary vasa recta. Banff lesion score “i-IFTA” evaluates the extent of inflammation in scarred cortex (including interstitial fibrosis and tubular atrophy).

FIG. 7 depicts the fluorescence-activated cell sorting (FACS) results of B cells, plasmablasts & plasma cells and B_{reg} cells from specimens before and after Daratumumab treatment on the patient (in the Example).

FIG. 8 depicts the FACS results of CD4+ cells, Tfh cells and T_{reg} cells from specimens before and after Daratumumab treatment on the patient (in the Example).

FIG. 9 depicts the FACS results of plasmablasts & plasma cells, B_{reg} cells and follicular Th cells from a normal control subject.

FIG. 10 shows representative microscopic images of renal biopsy on the patient (in the Example) before Daratumumab treatment. The patient has end-stage renal disease (ESRD), is secondary to diabetes, status post simultaneous pancreas and kidney transplant back in 2011, complicated by polyomavirus infection and subsequent graft failure in 2013, who is most recently status post living renal transplant in November 2017 and complicated by delayed graft function and positive donor specific antibodies.

FIG. 11 shows representative microscopic images of renal transplant biopsy on the patient (in the Example) after Daratumumab treatment.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“CD38” refers to the CD38 protein (synonyms: ADP-ribosyl cyclase 1, cADPr hydrolase 1, cyclic ADP-ribose hydrolase 1); and in various embodiments refers to the human CD38 protein. Human CD38 includes an amino acid sequence of:

(SEQ ID NO: 1)
MANCEFSPVSGDKPCCRLSRRAQLCLGVSILVLILVVVLAVVVPRWRQQW
SGPGTTKRFPTEIHPEMRHVDCQSVWDAFKGAFISKHPCNITEEDYQPLM
KLGTQTVPCNKILLWSRIKDLAHQFTQVQRDMFTLEDTLLGYLADDLTWC
GEFNTSKINYQSCPDWRKDCSNNPVSVFWKTVSRRFAEAACDVVHVMLNG
SRSKIFDKNSTFGSVEVHNLQPEKVQTLEAWVIHGGREDSRDLCQDPTIK
ELESIISKRNIQFSCKNIYRPDKFLQCVKNPEDSSCTSEI.

In various embodiments, the anti-CD38 agents are capable of killing a CD38+ cell by one or more means such as apoptosis, antibody-dependent cell-mediated cytotoxicity (ADCC), and complement-dependent cytotoxicity (CDC). In further embodiments, anti-CD38 agents are capable of binding CD38, thereby blocking, occupying and preventing its function in CD38+ cells.

The term “antibodies” as used herein is meant in a broad sense and includes immunoglobulin molecules including polyclonal antibodies, monoclonal antibodies including murine, human, human-adapted, humanized and chimeric monoclonal antibodies, antibody fragments, bispecific or multispecific antibodies, dimeric, tetrameric or multimeric antibodies, and single chain antibodies.

Immunoglobulins can be assigned to five major classes, namely IgA, IgD, IgE, IgG and IgM, depending on the heavy chain constant domain amino acid sequence. IgA and IgG are further sub-classified as the isotypes IgA₁, IgA₂, IgG₁, IgG₂, IgG₃ and IgG₄. Antibody light chains of any vertebrate species can be assigned to one of two clearly distinct types, namely kappa (κ) and lambda (λ), based on the amino acid sequences of their constant domains.

The term “antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (V_{H}), or a light chain variable region (V_{L}). Antibody fragments include a Fab fragment (an antigen-binding fragment, Fab), a monovalent fragment consisting of the V_{L}, V_{H}, C_{L} and C_{H1} domains; a F(ab)₂ fragment, a bivalent fragment comprising two Fab fragments linked by a disulfide bridge at the hinge region; a Fd fragment consisting of the V_{H} and C_{H1} domains; a Fv fragment consisting of the V_{L} and V_{H} domains of a single arm of an antibody; a domain antibody (dAb) fragment (Ward et al (1989) Nature 341:544-546), which consists of a V_{H} domain. V_{H} and V_{L} domains can be engineered and linked together via a synthetic linker to form various types of single chain antibody designs where the V_{H}/V_{L} domains pair intramolecularly, or intermolecularly in those cases when the V_{H} and V_{L} domains are expressed by separate single chain antibody constructs, to form a monovalent antigen binding site, such as single chain Fv (scFv) or diabody; described for example in PCT Intl. Publ. Nos. WO1998/44001, WO1988/01649, WO1994/13804, and WO1992/01047. These antibody fragments are obtained using well known techniques known to those of skill in the art, and the fragments are screened for utility in the same manner as are full length antibodies.

An antibody variable region consists of a “framework” region interrupted by three “antigen binding sites”. The antigen binding sites are defined using various terms such as Complementarity Determining Regions (CDRs), three in the V_{H} (HCDR1, HCDR2, HCDR3), and three in the V_{L} (LCDR1, LCDR2, LCDR3), are based on sequence variability (Wu and Kabat J Exp Med 132:211-50, 1970; Kabat et al Sequences of Proteins of Immunological Interest, 5th Ed. Public Health Service, National Institutes of Health, Bethesda, Md., 1991) or “Hypervariable regions”, “HVR”, or “HV”, three in the V_{H} (H1, H2, H3) and three in the V_{L} (L1, L2, L3), refer to the regions of an antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro, Mol Recognit 17:132-43, 2004). The International ImMunoGeneTics (IMGT) database provides a standardized numbering and definition of antigen-binding sites. The correspondence between CDRs, HVs and IMGT delineations is described in Lefranc et al., Dev Comparat Immunol 27:55-77, 2003.

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

“Humanized antibody” refers to an antibody in which the antigen binding sites are derived from non-human species and the variable region frameworks are derived from human immunoglobulin sequences. Humanized antibodies may include substitutions in the framework regions so that the framework may not be an exact copy of expressed human immunoglobulin or germline gene sequences.

“Human-adapted” antibodies or “human framework adapted (HFA)” antibodies refer to humanized antibodies adapted according to methods described in U.S. Pat. Publ. No. US2009/0118127. Human-adapted antibodies are humanized by selecting the acceptor human frameworks based on the maximum CDR and FR similarities, length compatibilities and sequence similarities of CDR1 and CDR2 loops and a portion of light chain CDR3 loops.

“Human antibody” refers to an antibody having heavy and light chain variable regions in which both the framework and the antigen binding sites are derived from sequences of human origin. If the antibody contains a constant region, the constant region also is derived from sequences of human origin.

A human antibody comprises heavy or light chain variable regions that are “derived from” sequences of human origin wherein the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. A “human antibody” may contain amino acid differences when compared to the human germline or rearranged immunoglobulin sequences due to for example naturally occurring somatic mutations or intentional introduction of substitutions in the framework or antigen binding sites. Typically, a human antibody is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by a human germline or rearranged immunoglobulin gene. In some cases, “human antibody” may contain consensus framework sequences derived from human framework sequence analyses, for example as described in Knappik et al., J Mol Biol 296:57-86, 2000), or synthetic HCDR3 incorporated into human immunoglobulin gene libraries displayed on phage, for example as described in Shi et al., J Mol Biol 397:385-96, 2010 and Intl. Pat. Publ. No. WO2009/085462. Antibodies in which antigen binding sites are derived from a non-human species are not included in the definition of human antibody.

The term “recombinant antibody” as used herein, includes all antibodies that are prepared, expressed, created or isolated by recombinant means, such as antibodies isolated from an animal (e.g., a mouse) that is transgenic or transchromosomal for human immunoglobulin genes or a hybridoma prepared therefrom (described further below), antibodies isolated from a host cell transformed to express the antibody, antibodies isolated from a recombinant, combinatorial antibody library, and antibodies prepared, expressed, created or isolated by any other means that involve splicing of human immunoglobulin gene sequences to other DNA sequences, or antibodies that are generated in vitro using Fab arm exchange such as bispecific antibodies.

The term “monoclonal antibody” as used herein refers to a preparation of antibody molecules of single molecular composition. A monoclonal antibody composition displays a single binding specificity and affinity for a particular epitope, or in a case of a bispecific monoclonal antibody, a dual binding specificity to two distinct epitopes.

The term “epitope” as used herein means a portion of an antigen to which an antibody specifically binds. Epitopes usually consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” as used herein refers to a polypeptide or a polynucleotide that differs from a reference polypeptide or a reference polynucleotide by one or more modifications for example, substitutions, insertions or deletions.

The terms “treat” or “treatment” refers to both therapeutic treatment and prophylactic or preventative measures, wherein the object is to prevent or slow down (lessen) an undesired physiological change or disorder, such as the development or spread of tumor or tumor cells. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

A “therapeutically effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction of a tumor burden, arrested or slowed growth of a tumor, and/or absence of metastasis of cancer cells to other locations in the body.

“HLA-sensitized (HS) patient” for kidney transplantation refers to patients awaiting kidney transplantation (e.g., on the United Network for Organ Sharing (UNOS) waitlist) whose calculated panel reactive antibodies (cPRA) or percentage of likely crossmatch incompatible donors is ≥50%, who in various embodiments also has demonstrable DSA using LUMINEX bead technology and a history of sensitizing events (previous transplants, blood transfusions and/or pregnancies). The presence of HLA specific antibodies can be determined by testing patient sera against cells from a panel of HLA typed donors or against solubilized HLA antigens attached to solid supports. Generally, HLA-sensitized patients refer to patients whose cPRA is no less than 10%, 20%, 30%, 40% or 50%.

A positive crossmatch (+CMX) indicates the presence of donor specific alloantibodies (DSA) in the serum of a potential recipient, and can be associated with a rate of graft loss that exceeds 80%.

A “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

The term “administering,” refers to the placement an agent as disclosed herein into a subject by a method or route which results in at least partial localization of the agents at a desired site.

The term “in combination with” as used herein means that two or more therapeutics can be administered to a subject together in a mixture, concurrently as single agents or sequentially as single agents in any order.

“Selectively binds” or “specifically binds” refers to the ability of an antibody or antibody fragment thereof described herein to bind to a target, such as a molecule present on the cell-surface, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

“Ineffective” treatment refers to when a subject is administered a treatment and there is no improvement or less than 1%, 5%, or 10% improvement in symptoms.

Organ transplantation remains the best option for patients with end-stage cardiac and renal disease. Highly-HLA sensitized patients have an immunologic barrier to life-saving transplantation. Past medical histories of pregnancy, blood transfusion and antecedent organ transplantation often lead to heightened anti-HLA antibody production due to the immunogenicity of dissimilar HLA class I and II epitopes. Additionally, intense immunologic memory is induced, resulting in long-term persistence of these allo-immune responses. Alloantibodies increase ABMR risk and reduce graft survival substantially. Consequently, patients are tested for the presence of high-titer HLA antibodies to identify more compatible organs. Patients with panel reactive HLA antibodies (PRA) >90% often have difficulty obtaining compatible allografts and remain on the wait list for extended periods of time, increasing morbidity and mortality. Current methods to reduce donor-specific HLA antibodies (DSAs), are only modestly successful. Rituximab is often used to target the CD20 protein found on B-lymphocytes as a means of desensitization. Yet the plasma cells (PCs) producing antibodies typically lack CD20 expression which may explain the limited success of this agent. Intravenous immunoglobulin (IVIg) and plasmapheresis (PLEX) are used but the former has limited efficacy in altering immune function and the latter is temporizing and often stimulates intense rebound antibody production.

ABMR is a severe, often unremitting form of rejection with a poor prognosis. The pathways of allo-sensitization resulting in DSA production that ultimately mediate ABMR are initiated by exposure to human cells and tissues. Briefly, allo-antigens from the donor are presented by antigen presenting cells (APCs) to naïve T-cells. Under the influence of IL-21 and IL-6, T-naïve cells mature into Tfh cells that activate naïve B-cells to mature into B-memory cells that evolve to IL-6 producing plasmablast (PB) and ultimately DSA producing, long-lived PCs. DSAs interact with the allograft primarily through complement dependent cytotoxicity (CDC) and antibody-mediated cytotoxicity (ADCC) to produce allograft injury and loss.

Initial efforts to target plasma cells in ABMR treatment have focused on the proteasome inhibitor, bortezomib, which is approved to treat multiple myeloma (MM) through plasma cell depletion. However, recent clinical trials in ABMR treatment and desensitization showed poor results with severe side effects and intense DSA rebound shortly after completion of therapy. The reasons for this likely relate to increases in T-follicular (Tfh) cell germinal center activity with generation of new B-cell responses from B-memory cells as humoral compensation for PC depletion.

Thus, there is an unmet medical need for development of novel desensitization agents that would improve access to life-saving transplants. New agents are also desperately needed to address ABMR. In this regard, daratumumab, a humanized IgGk monoclonal antibody targeting CD38, a protein found on antibody-producing plasmablasts (PBs) and plasma cells (PCs) may have significant advantages over bortezomib as it has the ability to deplete DSA-producing cells (CD38+ PB & PCs) without the significant adverse events or serious adverse events associated with proteasome inhibitors. Theoretically, this should result in reduction of HLA antibodies and possibly impact anamnestic responses with limited AE/SAEs.

### Methods and Systems

Various embodiments of methods of treating, reducing the likelihood or severity of antibody-mediated rejection of an organ transplant, and/or desensitizing an HLA-sensitized subject by removing donor specific antibodies, provide administering an anti-CD38 antibody or a pharmaceutical composition comprising an anti-CD38 antibody to the subject. Various embodiments provide the anti-CD38 antibody includes but is not limited to daratumumab or a variant that has identical heavy chain and/or light chain, or identical complementarity determining regions as detailed below. Further embodiments of the methods administering to the subject in need thereof one or more of isatuximab, MOR-202, GBR-1342, AMG-424, TAK-169, MT-4019ND, STI-6129, A-145D and EDC-8. Yet additional embodiments of the methods of treating, reducing the likelihood or severity of antibody-mediated rejection of an organ transplant, and/or desensitizing an HLA-sensitized subject, provide administering to the subject a therapy that targets CD38 or CD38-expressing cells, and the therapy that targets CD38 or CD38-expressing cells can be cell therapies such as CAR-T cell therapies, CAR-NK cell therapies, gene therapies, toxin bodies, fusion proteins with an CD38-binding domain (such as an anti-CD38 scFv) or a combination thereof.

Daratumumab (anti-CD38; HUMAX-CD38, JNJ-54767414, DARZALEX) is a humanized monoclonal with specificity for plasma cells and other immune cells designed for treatment of multiple myeloma. Daratumumab contains a heavy chain amino acid sequence of SEQ ID No: 2, a light chain amino acid sequence of SEQ ID No: 3; a variable heavy region (V_{H}) amino acid sequence of SEQ ID No: 4, a variable light region (V_{L}) amino acid sequence of SEQ ID No: 5; heavy chain complementarity determining regions (HCDR) 1 (HCDR1), 2 (HCDR2) and 3 (HCDR3) amino acid sequences of SEQ ID Nos: 6, 7 and 8, respectively; and light chain complementarity determining regions (LCDR) 1 (LCDR1), 2 (LCDR2) and 3 (LCDR3) amino acid sequences of SEQ ID Nos: 9, 10 and 11, respectively; and is of IgG1/κ subtype.

SEQ ID No: 2 is as shown:

EVQLLESGGG LVQPGGSLRL SCAVSGFTFN SFAMSWVRQA
PGKGLEWVSA ISGSGGGTYY ADSVKGRFTI SRDNSKNTLY
LQMNSLRAED TAVYFCAKDK ILWFGEPVFD YWGQGTLVTV
SSASTKGPSV FPLAPSSKST SGGTAALGCL VKDYFPEPVT
VSWNSGALTS GVHTFPAVLQ SSGLYSLSSV VTVPSSSLGT
QTYICNVNHK PSNTKVDKRV EPKSCDKTHT CPPCPAPELL
GGPSVFLFPP KPKDTLMISR TPEVTCVVVD VSHEDPEVKF
NWYVDGVEVH NAKTKPREEQ YNSTYRVVSV LTVLHQDWLN
GKEYKCKVSN KALPAPIEKT ISKAKGQPRE PQVYTLPPSR
EEMTKNQVSL TCLVKGFYPS DIAVEWESNG QPENNYKTTP
PVLDSDGSFF LYSKLTVDKS RWQQGNVFSC SVMHEALHNH
YTQKSLSLSP GK

SEQ ID No: 3 is as shown:

EIVLTQSPAT LSLSPGERAT LSCRASQSVS SYLAWYQQKP
GQAPRLLIYD ASNRATGIPA RFSGSGSGTD FTLTISSLEP
EDFAVYYCQQ RSNWPPTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

SEQ ID No: 4 is as shown:

EVQLLESGGG LVQPGGSLRL SCAVSGFTFN SFAMSWVRQA
PGKGLEWVSA ISGSGGGTYY ADSVKGRFTI SRDNSKNTLY
LQMNSLRAED TAVYFCAKDK ILWFGEPVFD YWGQGTLVTV
SS

SEQ ID No: 5 is as shown:

EIVLTQSPAT LSLSPGERAT LSCRASQSVS SYLAWYQQKP
GQAPRLLIYD ASNRATGIPA RFSGSGSGTD FTLTISSLEP
EDFAVYYCQQ RSNWPPTFGQ GTKVEIK

SEQ ID No: 6 is SFAMS
SEQ ID No: 7 is AISGSGGGTY YADSVKG
SEQ ID No: 8 is DKILWFGEPV FDY
SEQ ID No: 9 is RASQSVSSYL A
SEQ ID No: 10 is DASNRAT
SEQ ID No: 11 is QQRSNWPPTF

Various embodiments of the methods of reducing anti-HLA antibodies in a subject, desensitizing a subject for organ transplantation, and/or treating a subject with symptoms of ABMR include administering to the subject an effective amount of a composition comprising daratumumab to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of daratumumab to the subject. In some embodiments, a composition consisting essentially of daratumumab contains daratumumab, pharmaceutically acceptable excipient(s) and/or solvent(s), and optionally a fragment of daratumumab. In other embodiments, a composition consisting essentially of daratumumab contains daratumumab, or a fragment thereof, and pharmaceutically acceptable excipient(s) and/or solvent(s), but does not include another active ingredient such as bortezomib or carfilzomib.

Various embodiments of the methods of reducing anti-HLA antibodies in a subject, desensitizing a subject for organ transplantation, and/or treating a subject with symptoms of ABMR include administering to the subject an effective amount of a composition comprising an antibody that comprises (i) a variable heavy chain sequence that is 100% or about 99%, 98%, 97%, 96% 95%, 94%, 93%, 92%, 91%, 90%, 89%, 88%, 87%, 86%, 85%, 84%, 83%, 82%, 81%, or 80% identical to the polypeptide sequence set forth in SEQ ID NO:4, and (ii) a variable light chain sequence that is 100% or about 99%, 98%, 97%, 96% 95%, 94%, 93%, 92%, 91%, 90%, 89%, 88%, 87%, 86%, 85%, 84%, 83%, 82%, 81%, or 80% identical to the polypeptide sequence set forth in SEQ ID NO:5, to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of an antibody that comprises (i) a variable heavy chain sequence that is 100% or about 99%, 98%, 97%, 96% 95%, 94%, 93%, 92%, 91%, 90%, 89%, 88%, 87%, 86%, 85%, 84%, 83%, 82%, 81%, or 80% identical to the polypeptide sequence set forth in SEQ ID NO:4, and (ii) a variable light chain sequence that is 100% or about 99%, 98%, 97%, 96% 95%, 94%, 93%, 92%, 91%, 90%, 89%, 88%, 87%, 86%, 85%, 84%, 83%, 82%, 81%, or 80% identical to the polypeptide sequence set forth in SEQ ID NO:5, to the subject. In some embodiments, a composition consisting essentially of the antibody contains the antibody, pharmaceutically acceptable excipient(s) and/or solvent(s), and optionally a fragment of the antibody. In other embodiments, a composition consisting essentially of the antibody contains the antibody, or a fragment thereof, and pharmaceutically acceptable excipient(s) and/or solvent(s), but does not include another active ingredient such as bortezomib or carfilzomib.

Various embodiments of the methods of reducing anti-HLA antibodies in a subject, desensitizing a subject for organ transplantation, and/or treating a subject with symptoms of ABMR include administering to the subject an effective amount of a composition comprising isatuximab to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of isatuximab to the subject. In some embodiments, a composition consisting essentially of isatuximab contains isatuximab, pharmaceutically acceptable excipient(s) and/or solvent(s), and optionally a fragment of isatuximab. In other embodiments, a composition consisting essentially of isatuximab contains isatuximab, or a fragment thereof, and pharmaceutically acceptable excipient(s) and/or solvent(s), but does not include another active ingredient such as bortezomib or carfilzomib.

Various embodiments of the methods of reducing anti-HLA antibodies in a subject, desensitizing a subject for organ transplantation, and/or treating a subject with symptoms of ABMR include administering to the subject an effective amount of a composition comprising MOR-202 to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of MOR-202 to the subject. In some embodiments, a composition consisting essentially of MOR-202 contains MOR-202, pharmaceutically acceptable excipient(s) and/or solvent(s), and optionally a fragment of MOR-202. In other embodiments, a composition consisting essentially of MOR-202 contains MOR-202, or a fragment thereof, and pharmaceutically acceptable excipient(s) and/or solvent(s), but does not include another active ingredient such as bortezomib or carfilzomib. MOR-202 is an experimental antibody that binds to the CD38 surface molecule, and MOR-202 is under clinical investigation in relapsed or refractory multiple myeloma patients in a Phase ½a trial (NCT01421186). MOR-202 in the disclosed methods refers to the experimental antibody or a variant thereof. Sequences of MOR-202 are described in U.S. Pat. No. 8,088,896, which is incorporated by reference in its entirety.

Various embodiments of the methods of reducing anti-HLA antibodies in a subject, desensitizing a subject for organ transplantation, and/or treating a subject with symptoms of ABMR include administering to the subject an effective amount of a composition comprising mAh003 to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of mAh003 to the subject. In some embodiments, a composition consisting essentially of mAh003 contains mAh003, pharmaceutically acceptable excipient(s) and/or solvent(s), and optionally a fragment of mAh003. In other embodiments, a composition consisting essentially of mAh003 contains mAh003, or a fragment thereof, and pharmaceutically acceptable excipient(s) and/or solvent(s), but does not include another active ingredient such as bortezomib or carfilzomib. mAh003 is another anti-CD38 antibody, whose sequences are described in U.S. Pat. No. 7,829,693, which is hereby incorporated by reference in its entirety.

Another anti-CD38 antibody is mAb024, whose sequences are described in U.S. Pat. No. 7,829,693, which is hereby incorporated by reference in its entirety. Various embodiments of the methods of reducing anti-HLA antibodies in a subject, desensitizing a subject for organ transplantation, and/or treating a subject with symptoms of ABMR include administering to the subject an effective amount of a composition comprising mAb024 to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of mAb024 to the subject. In some embodiments, a composition consisting essentially of mAb024 contains mAb024, pharmaceutically acceptable excipient(s) and/or solvent(s), and optionally a fragment of mAb024. In other embodiments, a composition consisting essentially of mAb024 contains mAb024, or a fragment thereof, and pharmaceutically acceptable excipient(s) and/or solvent(s), but does not include another active ingredient such as bortezomib or carfilzomib.

Various embodiments of the methods of reducing anti-HLA antibodies in a subject, desensitizing a subject for organ transplantation, and/or treating a subject with symptoms of ABMR include administering to the subject an effective amount of a composition comprising GBR-1342 to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of GBR-1342 to the subject. In some embodiments, a composition consisting essentially of GBR-1342 contains GBR-1342, pharmaceutically acceptable excipient(s) and/or solvent(s), and optionally a fragment of GBR-1342. In other embodiments, a composition consisting essentially of GBR-1342 contains GBR-1342, or a fragment thereof, and pharmaceutically acceptable excipient(s) and/or solvent(s), but does not include another active ingredient such as bortezomib or carfilzomib. GBR-1342 is bi-specific monoclonal antibody which acts by targeting CD38 and CD3.

Various embodiments of the methods of reducing anti-HLA antibodies in a subject, desensitizing a subject for organ transplantation, and/or treating a subject with symptoms of ABMR include administering to the subject an effective amount of a composition comprising AMG-424 to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of AMG-424 to the subject. In some embodiments, a composition consisting essentially of AMG-424 contains AMG-424, pharmaceutically acceptable excipient(s) and/or solvent(s), and optionally a fragment of AMG-424. In other embodiments, a composition consisting essentially of AMG-424 contains AMG-424, or a fragment thereof, and pharmaceutically acceptable excipient(s) and/or solvent(s), but does not include another active ingredient such as bortezomib or carfilzomib. AMG-424 is a bispecific monoclonal antibody that targets tumor antigen binding domain CD38 and cytotoxic T-cell CD3 binding domain.

Various embodiments of the methods of reducing anti-HLA antibodies in a subject, desensitizing a subject for organ transplantation, and/or treating a subject with symptoms of ABMR include administering to the subject an effective amount of a composition comprising TAK-169 to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of TAK-169 to the subject. In some embodiments, a composition consisting essentially of TAK-169 contains TAK-169, pharmaceutically acceptable excipient(s) and/or solvent(s), and optionally a fragment of isatuximab. In other embodiments, a composition consisting essentially of TAK-169 contains TAK-169, or a fragment thereof, and pharmaceutically acceptable excipient(s) and/or solvent(s), but does not include another active ingredient such as bortezomib or carfilzomib. TAK-169 is an engineered toxin body that targets cells expressing CD38. Without being bound by a theory, the scFv fragment of TAK-169 binds to cells expressing CD38 and the toxin blocks protein synthesis by catalyzing depurination which results in ribosome inactivation and inhibition of protein synthesis.

Various embodiments of the methods of reducing anti-HLA antibodies in a subject, desensitizing a subject for organ transplantation, and/or treating a subject with symptoms of ABMR include administering to the subject an effective amount of a composition comprising MT-4019ND to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of MT-4019ND to the subject. In some embodiments, a composition consisting essentially of MT-4019ND contains MT-4019ND, pharmaceutically acceptable excipient(s) and/or solvent(s), and optionally a fragment of MT-4019ND. In other embodiments, a composition consisting essentially of MT-4019ND contains MT-4019ND, or a fragment thereof, and pharmaceutically acceptable excipient(s) and/or solvent(s), but does not include another active ingredient such as bortezomib or carfilzomib. MT-4019ND is an engineered toxin body with immunogenic B and CD4+ T cell epitopes replaced with MHC-I, CD8+ T cell epitopes through epitope class switching, and MT-4019ND targets CD38.

Various embodiments of the methods of reducing anti-HLA antibodies in a subject, desensitizing a subject for organ transplantation, and/or treating a subject with symptoms of ABMR include administering to the subject an effective amount of a composition comprising STI-6129 to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of STI-6129 to the subject. In some embodiments, a composition consisting essentially of STI-6129 contains STI-6129, pharmaceutically acceptable excipient(s) and/or solvent(s), and optionally a fragment of STI-6129. In other embodiments, a composition consisting essentially of STI-6129 contains STI-6129, or a fragment thereof, and pharmaceutically acceptable excipient(s) and/or solvent(s), but does not include another active ingredient such as bortezomib or carfilzomib. STI-6129 is a conjugate between an anti-CD38 antibody, or fragments thereof, and a toxin payload duostatin 5.

Various embodiments of the methods of reducing anti-HLA antibodies in a subject, desensitizing a subject for organ transplantation, and/or treating a subject with symptoms of ABMR include administering to the subject an effective amount of a composition comprising A-145D to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of A-145D to the subject. In some embodiments, a composition consisting essentially of A-145D contains A-145D, pharmaceutically acceptable excipient(s) and/or solvent(s), and optionally a fragment of A-145D. In other embodiments, a composition consisting essentially of A-145D contains A-145D, or a fragment thereof, and pharmaceutically acceptable excipient(s) and/or solvent(s), but does not include another active ingredient such as bortezomib or carfilzomib. A-145D is an attenuated anti-CD38-interferon alpha fusion protein which acts by targeting cells expressing CD38.

Various embodiments of the methods of reducing anti-HLA antibodies in a subject, desensitizing a subject for organ transplantation, and/or treating a subject with symptoms of ABMR include administering to the subject an effective amount of a composition comprising EDC-8 to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of EDC-8 to the subject. In some embodiments, a composition consisting essentially of EDC-8 contains EDC-8, pharmaceutically acceptable excipient(s) and/or solvent(s), and optionally a fragment of EDC-8. In other embodiments, a composition consisting essentially of EDC-8 contains EDC-8, or a fragment thereof, and pharmaceutically acceptable excipient(s) and/or solvent(s), but does not include another active ingredient such as bortezomib or carfilzomib. EDC-8 combines an anti-CD38 antibody with steroid, and acts by targeting CD38.

Various embodiments of the methods of reducing anti-HLA antibodies in a subject, desensitizing a subject for organ transplantation, and/or treating a subject with symptoms of ABMR include administering to the subject an effective amount of a composition comprising immune cells (e.g., T cells) that express anti-CD38 CAR to the subject. Various embodiments of the methods of reducing anti-HLA antibodies in a subject, desensitizing a subject for organ transplantation, and/or treating a subject with symptoms of ABMR include administering to the subject an effective amount of a composition consisting essentially of immune cells (e.g., T cells) that express anti-CD38 CAR to the subject. In some embodiments, a composition consisting essentially of immune cells (e.g., T cells) that express anti-CD38 CAR contains the immune cells and pharmaceutically acceptable excipient(s) and/or solvent(s). In other embodiments, a composition consisting essentially of immune cells (e.g., T cells) that express anti-CD38 CAR contains the immune cells and pharmaceutically acceptable excipient(s) and/or solvent(s), but does not include another active ingredient such as bortezomib or carfilzomib. In some embodiments, the immune cells can be engineered T cells. In some embodiments, the immune cells can be engineered natural killer (NK) cells.

Another embodiment is provided improving survival and function of a solid organ transplant and/or treating or reducing the severity of antibody-mediated rejection of the solid organ transplant in a subject, which includes, consists essentially of, or consists of administering to the subject an antibody or an antigen-binding fragment, said antibody or antigen-binding fragment contains a variable heavy region (V_{H}) of amino acid sequence of SEQ ID No: 4.

Yet another embodiment of improving survival and function of a solid organ transplant and/or treating or reducing the severity of antibody-mediated rejection of the solid organ transplant in a subject includes, consists essentially of, or consists of administering to the subject an antibody or an antigen-binding fragment, said antibody or antigen-binding fragment contains a variable light region (V_{L}) of amino acid sequence of SEQ ID No: 5.

Yet another embodiment of improving survival and function of a solid organ transplant and/or treating or reducing the severity of antibody-mediated rejection of the solid organ transplant in a subject includes, consists essentially of, or consists of administering to the subject an antibody or an antigen-binding fragment, said antibody or antigen-binding fragment contains a variable heavy region (V_{H}) of amino acid sequence of SEQ ID No: 4, or one that is about 99%, 98%, 97%, 96%, 95%, 94%, 93%, 92%, 91%, 90%, 89%, 88%, 87%, 86%, 85%, 84%, 83%, 82%, 81%, or 80% identical to the polypeptide sequence set forth in SEQ ID NO:4, and a variable light region (V_{L}) of amino acid sequence of SEQ ID No:5, or one that is about 99%, 98%, 97%, 96%, 95%, 94%, 93%, 92%, 91%, 90%, 89%, 88%, 87%, 86%, 85%, 84%, 83%, 82%, 81%, or 80% identical to the polypeptide sequence set forth in SEQ ID NO:5.

Methods are also provided for desensitizing a patient and improving survival and function of a solid organ transplant in the patient, who is typically HLA-sensitized and to whom standard-of-care treatment has failed, and the methods include, consist essentially of, or consist of administering to the patient an effective amount of daratumumab, an CD38-binding fragment of daratumumab, or an antibody containing a variable heavy region (V_{H}) of amino acid sequence of SEQ ID No: 4, a variable light region (V_{L}) of amino acid sequence of SEQ ID No: 5 or both, or one that is about 99%, 98%, 97%, 96%, 95%, 94%, 93%, 92%, 91%, 90%, 89%, 88%, 87%, 86%, 85%, 84%, 83%, 82%, 81%, or 80%, identical to the polypeptide sequence set forth in SEQ ID NO:4, 5 or both. Various aspects of the disclosed methods include, consist essentially of, or consist of reduced presence of HLA antibodies in the patients following administration of Daratumumab, or an antigen-binding fragment thereof, compared to a value obtained from the same patient prior to the administration.

Further embodiments provide a method of (1) treating or reducing the severity of antibody-mediated rejection of a solid organ transplant and/or (2) desensitizing a subject so as to reduce the level of anti-HLA antibodies, compared to a value obtained from the same subject prior to the desensitization step, where the method comprises or consists of administering to the subject in need thereof an anti-CD38 antibody or a CD38-binding fragment thereof. In some aspects of the method, the anti-CD38 antibody comprises heavy chain complementarity determining regions (HCDR) 1 (HCDR1), 2 (HCDR2) and 3 (HCDR3) sequences of SEQ ID NOs: 6, 7 and 8, respectively, and light chain complementarity determining regions (LCDR) 1 (LCDR1), 2 (LCDR2) and 3 (LCDR3) sequences of SEQ ID NOs: 9, 10 and 11, respectively; or one that contains CDRs that are about 99%, 98%, 97%, 96% 95%, 94%, 93%, 92%, 91%, 90%, 89%, 88%, 87%, 86%, 85%, 84%, 83%, 82%, 81%, or 80% identical to the polypeptide sequences set forth in SEQ ID NOs:6-11.

Antibodies specific for CD38, or anti-CD38 antibodies, are described in U.S. Pat. No. 8,877,899 (Morphosys AG), WO1999/62526 (Mayo Foundation) WO200206347 (Crucell Holland), US2002164788 (Jonathan Ellis), which are incorporated by reference in their entirety.

Various embodiments provide the disclosed methods exclude administering Mozobil or another anti-CXCR4 antibody.

One other anti-CD38 antibody for use in methods of treating, reducing the likelihood or severity of ABMR is isatuximab. Isatuximab is a monoclonal antibody targeting CD38, containing a variable heavy region (V_{H}) of SEQ ID NO: 12 and a variable light region (V_{L}) of SEQ ID NO: 13 (the CDRs are bolded and underlined).

One embodiment provides treating, reducing the likelihood or severity of antibody-mediated rejection of an organ transplant, and/or desensitizing an HLA-sensitized subject by removing donor specific antibodies, includes, consists essentially of or consists of administering isatuximab, an antibody containing V_{H} of SEQ ID NO: 12 and/or V_{L} of SEQ ID NO: 13, or an antibody containing HCDR1 (SEQ ID NO: 14), HCDR2 (SEQ ID NO: 15), HCDR3 (SEQ ID NO: 16), LCDR1 (SEQ ID NO: 17), LCDR2 (SEQ ID NO: 18) and LCDR3 (SEQ ID NO: 19) that are identical to those of Isatuximab. Another embodiment provides treating, reducing the likelihood or severity of antibody-mediated rejection of an organ transplant, and/or desensitizing an HLA-sensitized subject by removing donor specific antibodies, includes, consists essentially of or consists of administering an anti-CD38 antibody or an anti-CD38 binding fragment thereof, wherein the antibody contains V_{H} of a sequence that is 100% or about 99%, 98%, 97%, 96% 95%, 94%, 93%, 92%, 91%, 90%, 89%, 88%, 87%, 86%, 85%, 84%, 83%, 82%, 81%, or 80% identical to the polypeptide sequences set forth in SEQ ID NO: 12, as well as V_{L} of a sequence that is 100% or about 99%, 98%, 97%, 96% 95%, 94%, 93%, 92%, 91%, 90%, 89%, 88%, 87%, 86%, 85%, 84%, 83%, 82%, 81%, or 80% identical to the polypeptide sequences set forth in SEQ ID NO: 13. Further embodiments provide treating, reducing the likelihood or severity of antibody-mediated rejection of an organ transplant, and/or desensitizing an HLA-sensitized subject by removing donor specific antibodies, includes, consists essentially of or consists of administering isatuximab, an antibody containing CDRs that are about 99%, 98%, 97%, 96% 95%, 94%, 93%, 92%, 91%, 90%, 89%, 88%, 87%, 86%, 85%, 84%, 83%, 82%, 81%, or 80% identical to the polypeptide sequences set forth in SEQ ID NOs:14-19.

SEQ ID NO: 12
QVQLVQSGAE VAKPGTSVKL SCKASGYTFT DYWMQWVKQR
PGQGLEWIGT IYPGDGDTGY AQKFQGKATL TADKSSKTVY
MHLSSLASED SAVYYCARGD YYGSNSLDYW GQGTSVTVSS
SEQ ID NO: 13
DIVMTQSHLS MSTSLGDPVS ITCKASQDVS TVVAWYQQKP
GQSPRRLIYS ASYRYIGVPD RFTGSGAGTD FTFTISSVQA
EDLAVYYCQQ HYSPPYTFGG GTKLEIKRT

Further embodiments of the methods of treating, reducing the likelihood or severity of antibody-mediated rejection of an organ transplant, and/or desensitizing an HLA-sensitized subject provide administering to the subject a therapy selected from the group consisting of an anti-CD38 antibody or a CD38-binding fragment thereof, a chimeric antigen receptor (CAR) T-cell or NK-cell therapy, a gene therapy, a CD38-targeting engineered toxin body, or a combination thereof. In some embodiments, the CAR T-cell therapy contains a genetically engineered T-cell that expresses a CAR containing at least one anti-CD38 single-chain variable fragment (scFv). One exemplary CD38-targeting CAR T-cell therapy is UCART-38. In some embodiments, the gene therapy is a polynucleotide encoding the CAR containing at least one anti-CD38 scFv or is a vector comprising the polynucleotide thereof. A CD38-targeting toxin body can kill CD38-expressing cells via enzymatic destruction of ribosomes, wherein the CD38-targeting toxin body contains a catalytic subunit and an anti-CD38 scFv, and the catalytic subunit can be a de-immunized A subunit from Shiga-like toxin. Exemplary CD38-targeting toxin bodies suitable for one or more of the methods disclosed herein include TAK-169, MT-4019ND. Further embodiments provide that fusion proteins are suitable for administration to a subject in need thereof for desensitization and/or treating ABMR. Exemplary fusion proteins include A-145D, which is an attenuated anti-CD38-interferon alpha fusion protein, and TAK-573, which is a CD38-targeting IgG4 fused with attenuated interferon alpha. In some embodiments, an anti-CD38 antibody in combination with steroid is administered to the subject in need thereof in one or more of the methods disclosed herein.

Various aspects of the disclosed methods include reduced presence of HLA antibodies in the patients following administration of Daratumumab, a CD38-binding fragment thereof, or another anti-CD38 therapy, compared to a value obtained from the same patient prior to the administration. Further aspects of the disclosed methods include reducing the amount of class I anti-HLA antibodies, reducing or removing memory B cells, regulatory B cells, plasmablasts and/or plasma cells, in the subject following the administration. Yet additional aspects of the disclosed methods of treating or reducing the severity of ABMR in the subject include improving ABMR pathology scores in the subject following the administration. The anti-CD38 antibody, or another anti-CD38 therapy, can be administered before organ transplantation in the patient. The anti-CD38 antibody, or another anti-CD38 therapy, can be administered after organ transplantation in the patient.

### Patient/Subject Selection

Various embodiments of the disclosed treatment and desensitization methods include or further consists of selecting a subject having established drug (e.g., eculizumab)-resistant antibody-mediated rejection of an organ transplant or to whom standard-of-care/desensitization treatment (e.g., immunoglobulin administration (IVIG), rituximab administration, thymoglobulin administration, and optionally plasma exchange (PLEX); or PLEX in combination with bortezomib administration; or PLEX in combination with carfilzomib) has failed, where the methods for treating or desensitizing the subject by administering to the patient an effective amount of Daratumumab or another anti-CD38 antibody described in this application.

Other embodiments of the disclosed treatment and desensitization methods include selecting a subject who is anti-HLA sensitized and administering to the subject an effective amount of a composition disclosed herein, or the subject being anti-HLA sensitized is required in the methods.

Yet another embodiment of the disclosed methods provides the subject shows symptoms of, or is diagnosed with, one or more of antibody-mediated rejection, cell-mediated rejection, endarteritis, glomerulitis, severe tubulitis, and peritubular capillaritis. Typically such a subject is also anti-HLA sensitized and/or having established drug-resistant ABMR or to whom standard-of-care treatment is ineffective. One embodiment provides the subject in the methods before the administration has a donor-specific anti-HLA antibody amount that is more than 10%, 20%, 30%, 40%, 50% or more than a control subject, or that is at least 10,000, 11,000, 12,000 or 13,000 units of mean fluorescence intensity (MFI).

Further embodiments provide the subject in the methods is waiting for allograft transplants. Other embodiments provide the subject in the methods is after allograft transplants.

Some embodiments provide the subject in the methods does not have multiple myeloma. Other embodiments provide the methods further include selecting a subject that does not have multiple myeloma and that is in need of or has received a transplantation.

Dosage & Treatment Regimen

In one embodiment, a method for reducing donor-specific antibodies and HLA desensitization in a subject (e.g., human subject) includes, consists essentially of, or consists of, administering plasma exchange (or plasmapheresis) and an effective amount of IVIG (e.g., at about 1 g/kg of the subject, at about 2 g/kg of subject, for a maximum of 140 g), in combination with administering an effective amount of an anti-CD38 antibody (e.g., Daratumumab at an amount equivalent to about 16 mg intravenously/kg/week of a subject for at least 1, 2, 3, or 4 weeks).

In another embodiment, a method for treating, reducing the likelihood or severity of antibody-mediated rejection of an organ transplant (e.g., kidney, heart, liver, lungs, pancreas, intestines, skin or combinations thereof) in a subject (e.g., human subject) includes, consists essentially of, or consists of, administering an effective amount of an anti-CD38 antibody (e.g., Daratumumab at an amount equivalent to about 16 mg intravenously/kg/week of a subject for at least 1, 2, 3, or 4 weeks) before and/or after transplantation. In a further embodiment, this method includes or further consists of administering plasma exchange (or plasmapheresis) and/or an effective amount of IVIG.

Some embodiments of these methods provide further assaying the biopsy from the subject after administration of an anti-CD38 antibody, and confirming one or more of (A) a stabilized level of glomerular filtration rate (GFR) over time (e.g., less than 10%, 20%, or 30% variations across two, three, or four consecutive biopsies); (B) a low level (e.g., at less than 10%, 20% or 30%) of DSA compared prior to administration of the anti-CD38 antibody; and (C) significant reductions in circulating HLA class I and good reductions in class II with resolution of ABMR, compared with prior to the anti-CD38 treatment of the same subject. In some embodiments when none of the three parameters (A) (B) and (C) are met, the method further includes repeated administration of an anti-CD38 antibody (e.g., Daratumumab), until at least one, and preferably two or all three parameters (A) (B) and (C) are met.

The effective amount of an anti-CD38 antibody for a subject may be investigated or limited based on safety evaluations. Safety evaluations include medical interviews, recording of adverse events, physical examinations, blood pressure, and laboratory measurements. Subjects are generally evaluated for adverse events (all grades), serious adverse events, and adverse events requiring study drug interruption or discontinuation at each study visit for the duration of their participation in the study.

In some embodiments, the effective amounts of an anti-CD38 antibody (e.g. Daratumumab or another one disclosed in this application), can be in the range of about 0.1-1 mg/period or time, 1-10 mg/period, 10-50 mg/period, 50-100 mg/period, 100-150 mg/period, 150-200 mg/period, 100-200 mg/period, 200-300 mg/period, 300-400 mg/period, 400-500 mg/period, 500-600 mg/period, 600-700 mg/period, 700-800 mg/period, 800-900 mg/period, 900-1000 mg/period, 1000-1100 mg/period, 1100-1200 mg/period, 1200-1300 mg/period, 1300-1400 mg/period, 1400-1500 mg/period, 1500-1600 mg/period, 1600-1700 mg/period, 1700-1800 mg/period, 1800-1900 mg/period, 1900-2000 mg/period, 2000-2100 mg/period, 2100-2200 mg/period, 2200-2300 mg/period, 2300-2400 mg/period, 2400-2500 mg/period, 2500-2600 mg/period, 2600-2700 mg/period, 2700-2800 mg/period, 2800-2900 mg/period or 2900-3000 mg/period. A period is a day, a week, a month, or another length of time. One aspect is the anti-CD38 antibody (e.g., Daratumumab) is administered at a weekly, biweekly or monthly frequency of any of above-mentioned dosage per period.

In further embodiments, the effective amount of an anti-CD38 antibody (e.g. Daratumumab, or a polypeptide having V_{H} polypeptide containing HCDR1, HCDR2, and HCDR3 polypeptides which respectively are contained in SEQ ID NO: 6, 7, and 8, and/or having V_{L} polypeptide containing LCDR1, LCDR2, and LCDR3 polypeptides which respectively are contained in SEQ ID NO: 9, 10, and 11, suitable for administration in the disclosed methods, may be in the range of 0.01-0.05 mg/kg, 0.05-0.1 mg/kg, 0.1-1 mg/kg, 1-5 mg/kg, 5-10 mg/kg, 10-15 mg/kg, 15-20 mg/kg, 20-50 mg/kg, 50-100 mg/kg. In additional embodiments, the effective amount of an anti-CD38 antibody (e.g., Daratumumab) or a disclosed polypeptide is about 1-2 mg/kg, 2-3 mg/kg, 3-4 mg/kg, 4-5 mg/kg, 5-6 mg/kg, 6-7 mg/kg, 7-8 mg/kg, 8-9 mg/kg, 9-10 mg/kg, 10-11 mg/kg, 11-12 mg/kg, 12-13 mg/kg, 13-15 mg, 15-20 mg/kg or 20-25 mg/kg. In additional embodiments, the effective amount of the anti-CD38 antibody, or a disclosed polypeptide, is any one or more of about 100-125 mg, 125-150 mg, 150-175 mg, 160-170 mg, 175-200 mg, 155-165 mg, 160-165 mg, 165-170 mg, 155-170 mg, or combinations thereof, which may be administered over 1, 2, 3, 4, 5, 6, 7, 8, 9, 10, 11, 12, 13, 14, 15, 16, 17, or 18 doses where some are administered before, some after, and others at both before and after organ transplantation.

In various embodiments, the anti-CD38 antibody (e.g., Daratumumab) is administered at any one or more of the dosages described herein at least once 1-7 times per week, 1-7 times per month, or 1-12 times per year for 1 month, 2 months, 3 months, 4 months, 5 months 6 months, 7 months, 8 months, 9 months, 10 months, 11 months, 12 months, 14 months, 16 months, 18 months. In some embodiments, the antibody is administered for several months prior to and months or even years after transplant in order to prevent or reduce antibody mediated damage to the transplanted organ.

In various embodiments, the methods include intravenously administering an anti-CD38 antibody, an anti-CD38 antibody fragment, or a CD38-targeting therapy to a subject in need thereof. In further embodiments, the methods include subcutaneously administering an anti-CD38 antibody, an anti-CD38 antibody fragment, or a CD38-targeting therapy to a subject in need thereof.

Further embodiments of the methods, as above described, provide that the patient is monitored before, at the time of, and/or after administration of a dose of the anti-CD38 antibody or disclosed composition. In one aspect, patient is monitored for clinical signs of rejection such as increases in serum creatinine and/or proteinuria, or decreases in eGFR in kidney transplants), or development of new DSA (de novo DSA). In one aspect, patient is monitored for histological signs of organ rejection or biopsy evidence of organ damage. In a further aspect, if signs of rejection, decrease in transplant function, and/or development of new DSA occur, the methods including administering a subsequent dose of the anti-CD38 antibody or a disclosed composition, or continuing the administration of the anti-CD38 antibody or disclosed composition. In yet another aspect, the methods provide that the anti-CD38 antibody or a disclosed composition is administered until no signs of rejection, decrease in transplant function, or new DSA are observed.

The foregoing treatments may be effected in combination with one or more other immunosuppressant regimens or other desensitization procedures.

### Combination Therapy

Further methods are provided of treating, reducing the likelihood or severity of ABMR where an effective amount of Daratumumab and an effective amount of a T cell-specific agent are administered to the subject. T cell-specific agents include tacrolimus and cellcept.

Other embodiments provide that the methods for desensitization and/or treatment of ABMR further include administering one or more anti-infectious agents, preferably post-transplantation, as a prophylaxis or therapeutics against bacterial, viral or fungal infections.

Exemplary anti-infectious agents suitable for use in the disclosed methods include antibiotics such as aminoglycosides (e.g., amikacin, gentamicin, kanamycin, neomycin, netilmicin, streptomycin, tobramycin, paromomycin), ansamycins (e.g., geldanamycin, herbimycin), carbacephems (e.g., loracarbef), carbapenems (e.g., ertapenem, doripenem, imipenem, cilastatin, meropenem), cephalosporins (e.g., first generation: cefadroxil, cefazolin, cefalotin or cefalothin, cefalexin; second generation: cefaclor, cefamandole, cefoxitin, cefprozil, cefuroxime; third generation: cefixime, cefdinir, cefditoren, cefoperazone, cefotaxime, cefpodoxime, ceftazidime, ceftibuten, ceftizoxime, ceftriaxone; fourth generation: cefepime; fifth generation: ceftobiprole), glycopeptides (e.g., teicoplanin, vancomycin), macrolides (e.g., azithromycin, clarithromycin, dirithromycin, erythromycin, roxithromycin, troleandomycin, telithromycin, spectinomycin), monobactams (e.g., aztreonam), penicillins (e.g., amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin), antibiotic polypeptides (e.g., bacitracin, colistin, polymyxin b), quinolones (e.g., ciprofloxacin, enoxacin, gatifloxacin, levofloxacin, lomefloxacin, moxifloxacin, norfloxacin, ofloxacin, trovafloxacin), rifamycins (e.g., rifampicin or rifampin, rifabutin, rifapentine, rifaximin), sulfonamides (e.g., mafenide, prontosil, sulfacetamide, sulfamethizole, sulfanilamide, sulfasalazine, sulfisoxazole, trimethoprim, trimethoprim-sulfamethoxazole (co-trimoxazole, “tmp-smx”), and tetracyclines (e.g., demeclocycline, doxycycline, minocycline, oxytetracycline, tetracycline) as well as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin combination, and tinidazole.

### Pharmaceutical Composition & Uses Thereof

In various embodiments, the present invention provides a pharmaceutical composition. In some embodiments, the pharmaceutical composition includes (1) Daratumumab; an antibody containing heavy chain of SEQ ID NO: 2 and/or light chain of SEQ ID NO: 3; an antibody containing variable heavy region of SEQ ID NO: 4 and a variable light region of SEQ ID NO: 5; or a polypeptide having V_{H} polypeptide containing HCDR1, HCDR2, and HCDR3 polypeptides which respectively are contained in SEQ ID NO: 6, 7 and 8 and having V_{L} polypeptide containing LCDR1, LCDR2, and LCDR3 polypeptides which respectively are contained in SEQ ID NO: 9, 10 and 11; and (2) a pharmaceutically acceptable carrier, such as pharmaceutically acceptable excipients.

In other embodiments, the pharmaceutical composition includes (1) an anti-CD38 antibody or antibody fragment thereof, or a CD38-targeting therapy, and (2) a pharmaceutically acceptable carrier, such as pharmaceutically acceptable excipients.

The pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to amino acids, starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In one embodiment, the disclosed methods involve administering a pharmaceutical composition which includes L-histidine, L-histidine monohydrochloride, sorbitol, polysorbate-80, and water for injection, and Daratumumab; an antibody containing heavy chain of SEQ ID NO: 2 and/or light chain of SEQ ID NO: 3; an antibody containing variable heavy region of SEQ ID NO: 4 and a variable light region of SEQ ID NO: 5; or a polypeptide having V_{H} polypeptide containing HCDR1, HCDR2, and HCDR3 polypeptides which respectively are contained in SEQ ID NO: 6, 7 and 8 and having V_{L} polypeptide containing LCDR1, LCDR2, and LCDR3 polypeptides which respectively are contained in SEQ ID NO: 9, 10 and 11.

In various embodiments, the pharmaceutical compositions according to the invention may be formulated for delivery via any route of administration. In one embodiment, the pharmaceutical composition is administered intravenously or subcutaneously to the subject. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

The pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion. Pharmaceutically acceptable solid or liquid carriers may be added to enhance or stabilize the composition, to facilitate preparation of the composition, or to provide sustained or controlled release (or increase the half-life) of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. Emulsion carriers include liposomes, or controlled release polymeric nanoparticles known in the art. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

In some embodiments, before administration to patients, formulants may be added to Daratumumab; an antibody containing heavy chain of SEQ ID NO: 2 and/or light chain of SEQ ID NO: 3; an antibody containing variable heavy region of SEQ ID NO: 4 and a variable light region of SEQ ID NO: 5; or a polypeptide having V_{H} polypeptide containing HCDR1, HCDR2, and HCDR3 polypeptides which respectively are contained in SEQ ID NO: 6, 7 and 8 and having V_{L} polypeptide containing LCDR1, LCDR2, and LCDR3 polypeptides which respectively are contained in SEQ ID NO: 9, 10 and 11.

In some embodiments, formulants may be added to another anti-CD38 therapy, such as daratumumab, isatuximab, MOR-202, GBR-1342, AMG-424, TAK-169, MT-4019ND, STI-6129, A-145D, EDC-8, immune cells that express anti-CD38 CAR, or polynucleotide therapy thereof.

A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

### Kits

In various embodiments, the present invention provides a kit for identification and desensitization or treating ABMR in organ transplant recipients. The kit is an assemblage of materials or components, including an anti-CD38 antibody/antibody fragment/therapy (e.g., Daratumumab, Isatuximab, or another one disclosed in this application); an instruction or manual for identification and/or administration for desensitization and/or treatment before and after organ transplantation; one or more vessels as containers; optionally one or more diluents; and optionally a biological sample collect vessel.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat or inhibit anti-HLA antibodies or antibody-mediated rejection in a subject. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of an inventive composition containing daratumumab; an antibody containing heavy chain of SEQ ID NO: 2 and/or light chain of SEQ ID NO: 3; an antibody containing variable heavy region of SEQ ID NO: 4 and a variable light region of SEQ ID NO: 5; or a polypeptide having V_{H} polypeptide containing HCDR1, HCDR2, and HCDR3 polypeptides which respectively are contained in SEQ ID NO: 6, 7 and 8 and having V_{L} polypeptide containing LCDR1, LCDR2, and LCDR3 polypeptides which respectively are contained in SEQ ID NO: 9, 10 and 11; or an anti-CD38 antibody or antibody fragment. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Treatment of Antibody Mediated Rejections in Highly-HLA Sensitized Patients

The patient was a 33 year-old highly-HLA sensitized female who developed severe ABMR post-HLAi transplant and was resistant to treatment with PLEX+IVIg+Rituximab, thymoglobulin and eculizumab. She was diagnosed with end stage renal disease (ESRD), secondary to diabetes, status post simultaneous pancreas and kidney transplants in 2011, and was complicated by polyomavirus infection and subsequent graft failure in 2013. She was most recently status post living related renal transplant in November 2017, complicated by delayed graft function and positive DSA. Previous biopsy performed in November 2017 and January 2018 revealed features of acute calcineurin inhibitor toxicity and cell-mediated rejection, Banff '97 1A respectively. The patient was now being biopsied for positive DSA and markedly elevated serum creatinine (peak 5.5 g/D1). The patient demonstrated a DSA (DQ5) at MFI 13,000, unresponsive to previous treatments. After informed consent, the patient was treated with daratumumab intravenously (16 mg/kg weekly×4). Prior to and at completion of treatment, LUMINEX-HLA antibodies (class I & II) and immune cell phenotyping (CD4+, B-naive, B-memory, regulatory B-cells (B-regs), PB and PCs) were determined. Biopsies were performed pre and post-daratumumab treatment and analyzed using Banff 2017 criteria.

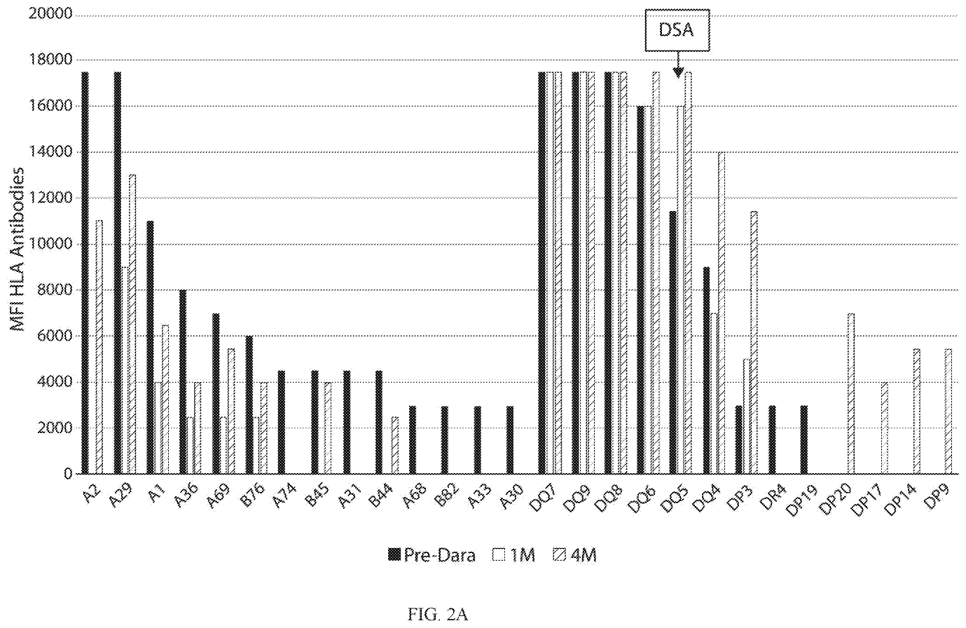

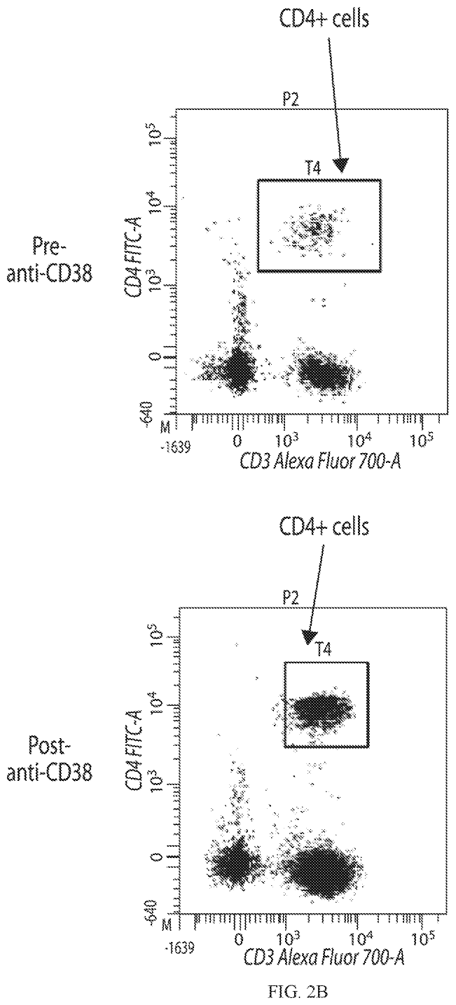

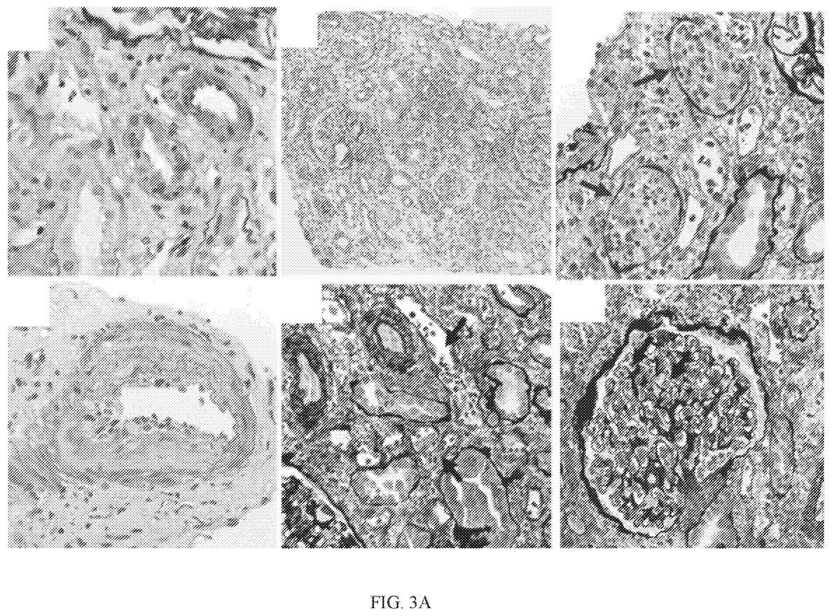

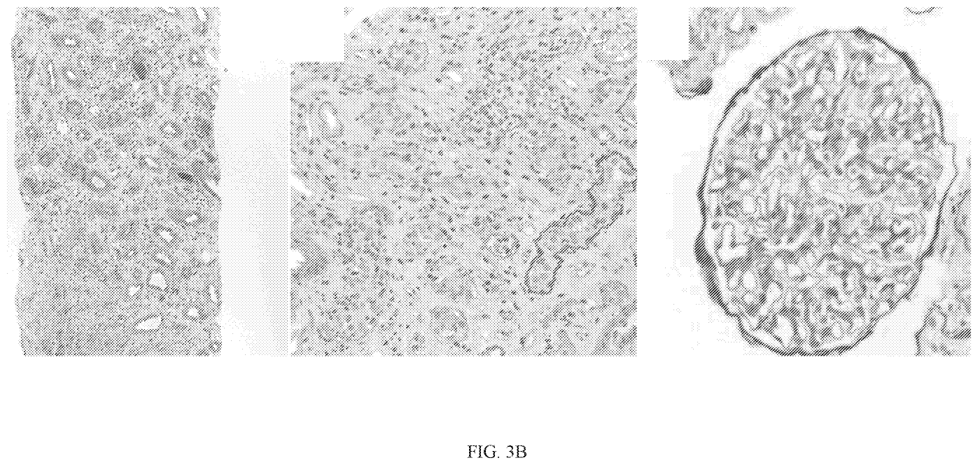

Results—there were no significant AE/SAEs associated with anti-CD38 use. Analysis of total HLA and DSAs is shown in FIG. 2A. Briefly, significant reductions in class I, but not class II, were seen with daratumumab therapy. DSA to DQ5 was not reduced and actually increased after daratumumab therapy. Additionally, flow cytometry analysis of T- and B-cell subsets showed complete elimination of B-memory, B-reg, PB and PCs similar to that seen in patient in Example 2. However, CD4+ T cells increased post-daratumumab (FIG. 2B). Renal biopsies were performed before and at completion of daratumumab (FIGS. 3A & 3B). Briefly, the original biopsy showed evidence of CMR (Banff 1A) which was treated with pulse steroids. Despite treatment, patient failed to respond and underwent a repeat biopsy one month later. Here, we see features of CMR (Banff 1A) and active ABMR C4d negative with peritubular capillaritis and glomerulitis. A third biopsy was performed at completion of daratumumab and showed ABMR resolution but was notable for intense T-cell rejection (Banff 1B). The patient also had repeat HLA antibody analysis at several time points (FIG. 2A). After daratumumab therapy, HLA class I antibodies showed a persistent decline, however a rebound of HLA class II antibodies was observed. In addition, there was no impact on the DQ5 DSA, despite improvements in ABMR pathology scores. Finally, the patient did not show meaningful improvements in renal function and returned to dialysis in June 2019.

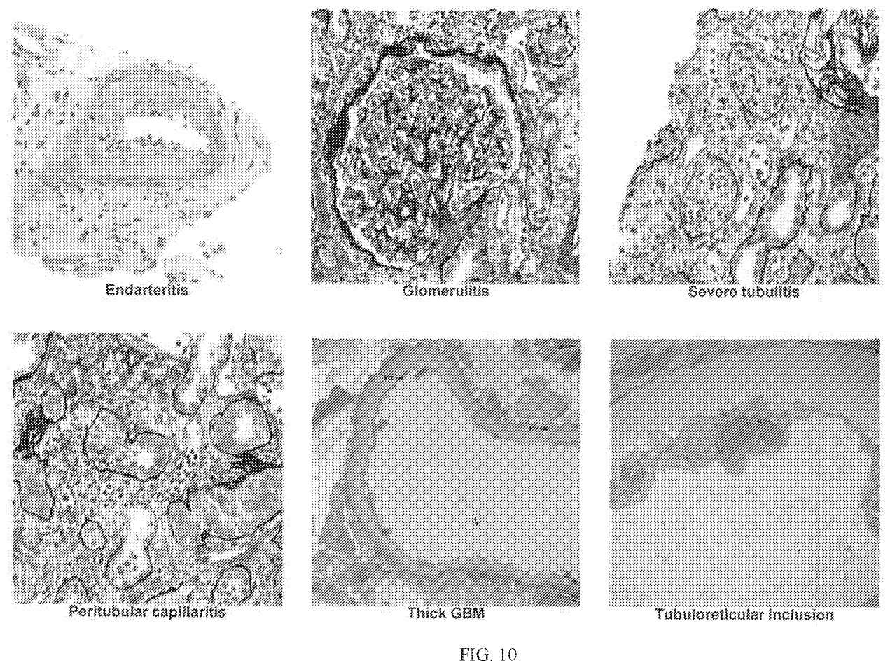

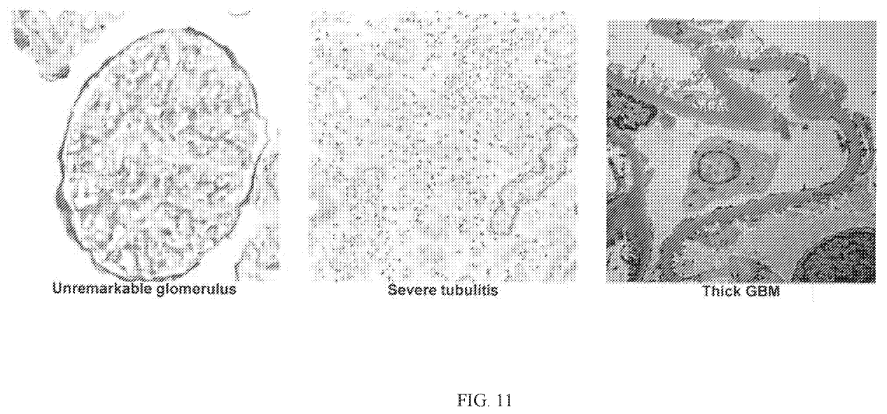

Before Daratumumab treatment, renal biopsy (FIG. 10) of the patient showed:

1. acute cell-mediated tubulointerstitial and vascular rejection (Banff '97 2A);
2. acute antibody-mediated rejection, C4d negative (comments detailed below);
3. diffuse diabetic glomerulosclerosis;
4. moderate inflamed interstitial fibrosis and tubular atrophy (IF/TA).

There were peritubular capillaritis and focal glomerulitis, along with the positive DSA. The findings were consistent with C4d negative antibody mediated rejection. The significance of frequent tubuloreticular structures/inclusions (TRIs) was uncertain. TRIs are most often seen in the setting of lupus nephritis, viral infections, and interferon therapy. However, recent publications reported the presence of TRIs in renal allografts and indicated an association with viral infections, previous rejection episodes, and the presence Class I HLA DSAs. There was approximately 5% global glomerulosclerosis with moderate inflamed parenchymal scarring.

Microscopic findings of the patient (before Daratumumab treatment) were as follows. The specimen for conventional light microscopy was studied with hematoxylin and eosin, periodic acid-Schiff, periodic acid-methenamine silver, and Masson's trichrome staining. The specimen consisted of two fragments of renal cortex, one which contained a portion of thickened capsule. Fourteen glomeruli were identified, one of which was globally sclerotic. Glomeruli were normal in size with single contoured capillary basement membranes and predominantly patent capillary lumina. While there were increased circulating intracapillary leukocytes, two glomeruli showed segmental occlusive endocapillary hypercellularity with swollen endothelial cells (glomerulitis). Mesangial areas displayed segmental, mild expansion by PAS and silver positive material without significant hypercellularity. No basement membrane double contours, segmental sclerosis, or crescents were seen. There was a diffuse mixed interstitial inflammatory infiltrate involving lymphocytes, plasma cells, and segmented leukocytes, which involved the entire cortex. The interstitial inflammation also involved areas of moderate tubular atrophy and interstitial fibrosis involving approximately 30% of the cortex. The inflammatory cells multifocally crossed tubular basement membranes to produce foci of severe tubulitis (greater than 24 leukocytes per 10 tubular epithelial cells), focally associated with tubular basement membrane rupture. One artery displayed swollen endothelial cells with superficial, undermining leukocytes (endarteritis). Few arterioles also contained reactive, swollen endothelial cells with adherent leukocytes. Approximately 70% to 80% of peritubular capillaries showed severe peritubular capillaritis (up to 16 leukocytes per peritubular capillary profile). Immunohistochemical staining for polyomavirus was performed with a cross-reactive against SV40 (with appropriate controls) and was negative.

Immunofluorescence microscopy was performed on frozen sections, with appropriate controls, stained with fluoresceinated antisera to human lgG, lgA, lgM, C1q, C3, albumin, fibrin and kappa and lambda immunoglobulin light chains and was graded on a scale of 0-4+. Each frozen section consisted of renal cortex with overlying capsule containing nine glomeruli, none of which were globally sclerotic. There was no significant glomerular or interstitial staining for immune reactants. C4d (stained using indirect immunofluorescence with appropriate controls) was negative in peritubular capillaries. The specimen for electron microscopy, studied first by light microscopy of methylene blue stained one micron thick sections, consisted of two fragments of renal cortex containing a single preserved glomerulus. The glomerulus had single-contoured capillary basement membranes and patent capillary lumina. Mesangial areas were unremarkable; no hypercellularity, segmental sclerosis, or crescents were seen. There was diffuse interstitial edema associated with a diffuse mixed interstitial inflammatory infiltrate composed of lymphocytes, plasma cells, and segmented leukocytes, multifocally associated with foci of severe tubulitis. Proximal tubules showed acute injury and epithelial cell necrosis. Vascular sampling was limited to arterioles which are unremarkable. Eighty-percent of peritubular capillaries showed at least moderate peritubular capillaritis. Ultrastructural analysis performed on a single glomerulus revealed glomerular capillary basement membranes with normal trilaminar structure and global thickening (up to 973 nm). Rare capillary loops showed very early, focal subendothelial electron lucent widening with glomerular basement membrane duplication. A single tubuloreticular inclusion was seen within endothelial cell cytoplasm. Mesangial areas showed expansion by matrix material without active electron dense (immune complex) deposits. Podocytes displayed partial (approximately 30%) foot process effacement. Peritubular capillary basement membranes were at most double-contoured and the other tubulointerstitial and vascular findings are confirmed. Table 1 summarizes the Banff scores of the specimen before Daratumumab treatment.

After consent, the patient was treated with anti-CD38 (Daratumumab at 16 mg/kg weekly×4). Prior to and at completion of treatment, LUMINEX-HLA antibodies HLA (class I &II) and immune cell phenotyping (T_{fh}, T_{reg}, Bnaive, plasmablast and plasma cells) were determined. A biopsy was also performed pre and post-Daratumumab treatment.

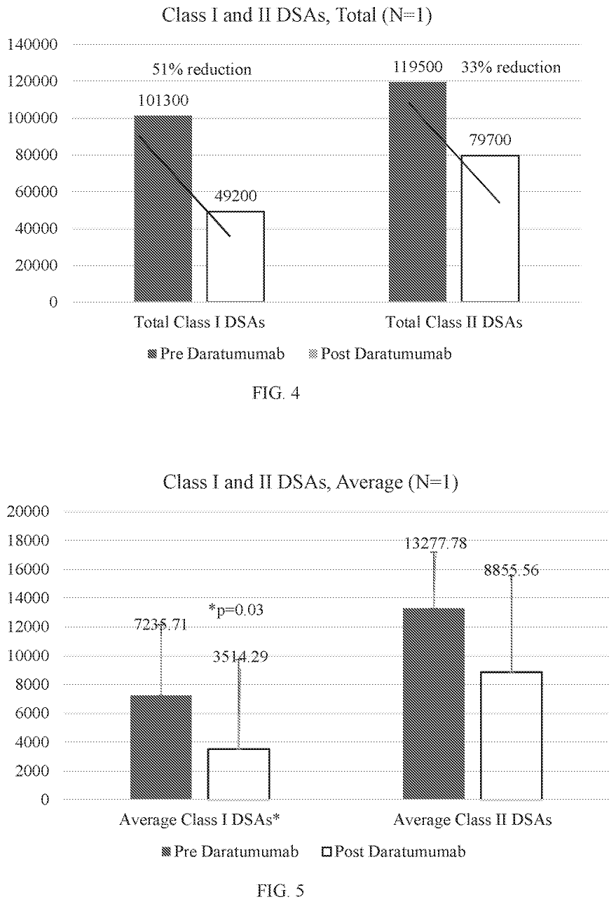

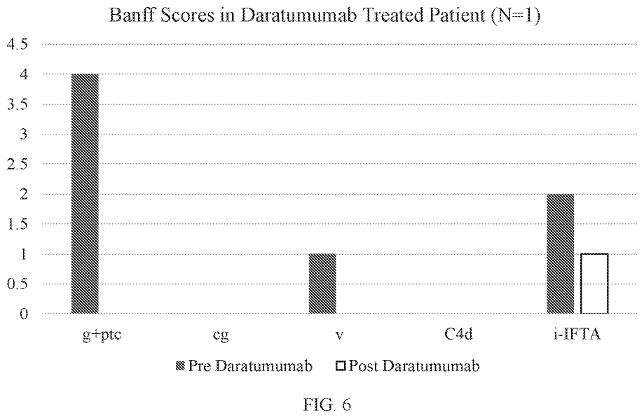

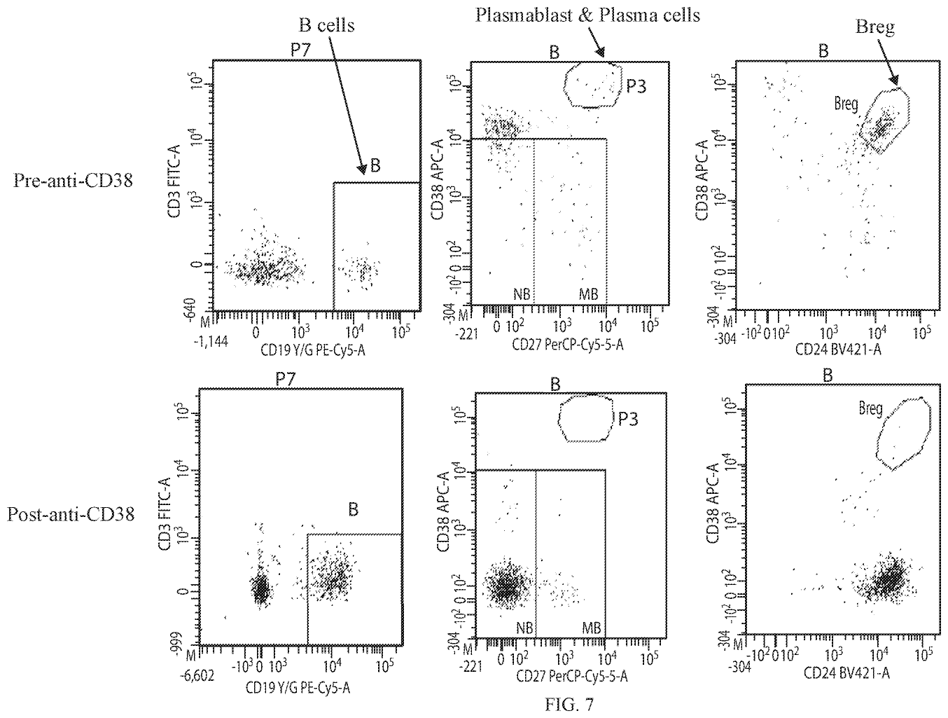

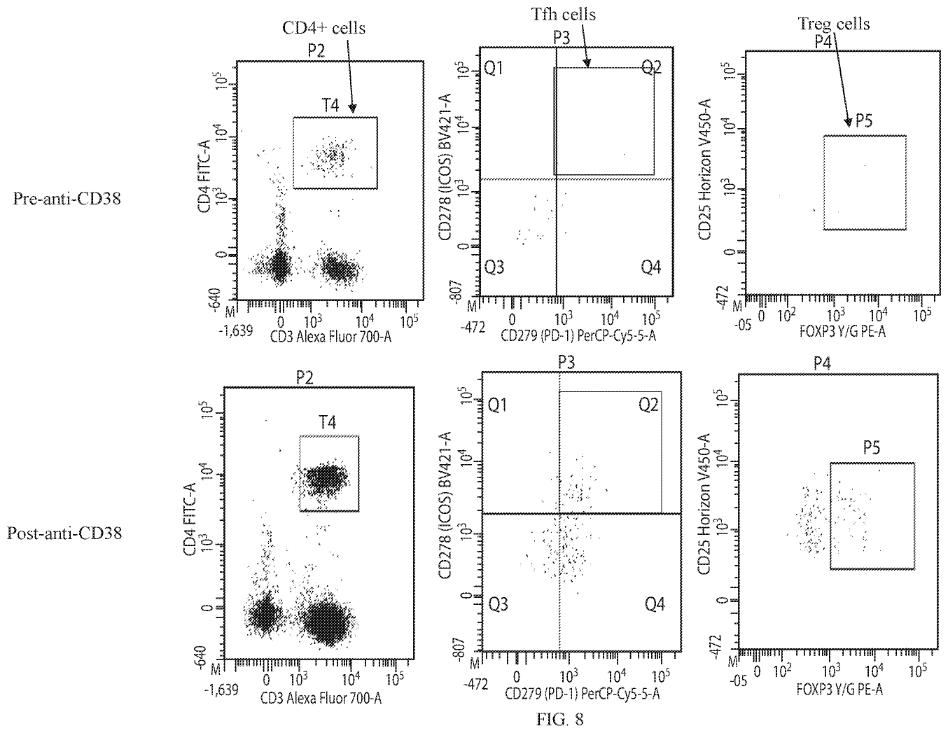

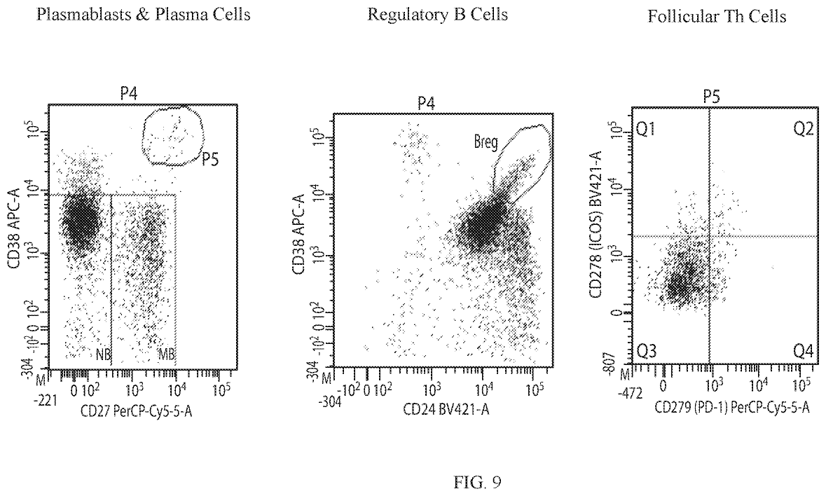

Pre- and post-transplant HLA antibody MFIs are shown in FIGS. 4 and 5. Briefly, HLA class I antibodies were reduced 51% (p=0.03) while class II were reduced 33% (p=NS). DSAs DQ7 were reduced from greater than 17,500 to about 5000 MFI. Immune cell phenotyping showed complete eradication of CD38+ cells including B_{regs}, B_{memory}, plasmablast, and plasma cells (compare FIG. 7 with FIG. 9). However, marked increases in total CD4+, T_{fh} and T_{reg} were seen in peripheral blood (compare FIG. 8 with FIG. 9). Repeat biopsy performed at completion of Daratumumab treatment showed no evidence of ABMR (FIG. 6, which summarizes the scores before Daratumumab treatment, shown in Table 1, and after Daratumumab treatment, shown in Table 2).

TABLE 1
Banff scores of the patient before Daratumumab treatment
Banff Scores

| | | | |
|---|---|---|---|
| t 3 | v 1 | cg 0 | i-lFTA 2 |
| i 3 | g 1 | ci 2 | ti 3 |
| ptc 3 | C4d (IF) 0 | ct 2 | pvl 0 |

TABLE 2
Banff scores of the patient after Daratumumab treatment
Banff Scores

| | | | |
|---|---|---|---|
| t 3 | v 0 | cg 0 | i-lFTA 1 |
| i 3 | g 0 | ci 1 | ti 3 |
| ptc 0 | C4d (IF) 0 | ct 1 | pvl 0 |

After Daratumumab treatment, renal transplant biopsy (FIG. 11) showed:

1. chronic active cell mediated rejection, Banff '97 1B;
2. features of mild diabetic glomerulosclerosis.

There was no evidence of active or chronic antibody mediated rejection. There was approximately 5% global glomerulosclerosis. While established parenchymal scarring was mild, there was extensive early tubular atrophy and organizing fibrosis.

Microscopic findings of the patient (after Daratumumab treatment) were as follows. The specimen for conventional light microscopy were studied with hematoxylin and eosin, periodic acid-Schiff, periodic acid-methenamine silver, and Masson's trichrome staining. Sections consisted of 1 fragment of renal cortex containing 12 glomeruli, 1 of which was globally sclerotic. Glomeruli were normal in size with single contoured capillary basement membranes and patent capillary lumina. Mesangial areas displayed segmental minimal expansion without significant hypercellularity. No segmental sclerosis or crescents were seen. Proximal tubules displayed acute injury and focal necrosis. There was a diffuse mixed interstitial inflammatory infiltrate which involved 90% of the cortex overall. This included 20% which showed well-established tubular atrophy and interstitial fibrosis and a superimposed component of extensive early tubular atrophy and organizing fibrosis was also seen. The inflammatory cells multifocally crossed tubular basement membranes to produce foci of severe (up to 30 leukocytes per 10 tubular epithelial cells) tubulitis. No nuclear viral inclusions or viral cytopathic effect was seen. Few small arteries and arterioles displayed muscular hypertrophy. Peritubular capillaries in non-inflamed areas were devoid of significant intraluminal inflammation. Within inflamed areas, the degree of peritubular capillaritis appeared proportional to the degree of surrounding interstitial inflammation. No endarteritis was seen. Immunofluorescence microscopy was performed on frozen sections, with appropriate controls, stained with fluoresceinated antisera to human lgG, lgA, lgM, C1q, C3, albumin, fibrin and kappa and lambda immunoglobulin light chains and was graded on a scale of 0-4+. Each frozen section consisted of renal cortex containing 4 glomeruli, none of which were globally sclerotic. There was no significant glomerular staining for immune reactants. Tubular casts stained for lgG (2+), lgA (4+), C3 (3+), kappa (3+) and lambda (3+) light chains. C4d (stained using indirect immunofluorescence with appropriate controls) was negative in peritubular capillaries. The remaining immune reactants were negative. The specimen for electron microscopy, studied first by light microscopy of methylene blue stained one micron thick sections, consisted of 1 fragment of renal cortex containing 2 glomeruli, neither of which was globally sclerotic. Glomeruli were histologically unremarkable; no hypercellularity, segmental sclerosis, or crescents were seen. Proximal tubules displayed acute necrosis. There was a diffuse mixed interstitial inflammatory infiltrate which was multifocally associated with foci of severe tubulitis. Vascular sampling was limited to arterioles which were unremarkable. Peritubular capillaries were largely devoid of significant intraluminal inflammation. Ultrastructural analysis performed on a single glomerulus revealed glomerular capillary basement membranes with normal trilaminar structure and global thickening (up to 681 nm). Although there were segmental subendothelial lucencies, no diagnostic features of transplant glomerulopathy were seen nor were tubuloreticular inclusions present within endothelial cell cytoplasm. Mesangial areas displayed minimal expansion by matrix material without active electron dense (immune complex) deposits. Podocytes displayed partial (approximately 20%) foot process effacement. Peritubular capillary basement membranes were single or double contoured. The other tubulointerstitial and vascular findings were confirmed.

Overall, Daratumumab was well tolerated with minimal adverse events noted. The treatment regimen showed significant reductions in circulating HLA class I and good reductions in class II with resolution of ABMR. However, elimination of B_{regs} may have incited CMR. Complete elimination of features of ABMR is conceived to be aided by anti-CD38 depletion of CD38+ NK cells, thus restricting antibody-dependent cellular cytotoxicity (ADCC).

### Example 2. Desensitization in Highly-HLA Sensitized Patients

A 21-year old male patient with congenital heart disease was referred to Cedars-Sinai Medical Center, for desensitization pending orthotopic heart transplantation (OHT). The patient had a life-long history of multiple cardiac surgeries and blood product exposure resulting in HLA sensitization (cPRA, 100%). Desensitization attempts at the referring facility using IVIg and rituximab were unsuccessful.

Upon admission, the patient was in overt heart failure. Evaluation by the heart transplant team determined he was an acceptable candidate for OHT pending successful desensitization. Prior to daratumumab use, HLA alloantibody desensitization with PLEX+bortezomib was attempted. Despite only four doses of bortezomib, he developed grade 3 sensory neuropathy without impact on HLA antibodies. In a second attempt, the proteasome inhibitor, carfilzomib+PLEX was used. Unfortunately, the patient developed life-threatening capillary leak syndrome after one dose of carfilzomib. After receiving informed consent from the patient and family, we offered treatment with daratumumab. The patient underwent 5 sessions of PLEX followed by IVIG (lg/kg) and daratumumab (16 mg/kg intravenously weekly×4).

Prior to PLEX+bortezomib therapy, the patient's PRA was 99% and 93% against HLA class I and II antigens, respectively. Class I C1q binding PRA was 53%. Treatment with PLEX+bortezomib did not impact HLA class I and II antibodies as PRA was 98% and 94%, respectively and the HLA class I specific C1q binding antibodies rose to 79%.

However, at the completion of four doses of daratumumab, the class I and II HLA antibodies were 86% and 93% with no C1q+ antibodies. Further analysis of the MFI of each HLA antibody specificity showed clinically significant reductions or elimination of most antibodies except for those with the highest MFI (>17,500). There were no significant AE/SAEs directly associated with daratumumab therapy. Based on these results, patient would have been able to receive a heart transplant after daratumumab. Unfortunately, the patient passed away from heart failure complications before a suitable organ was available.

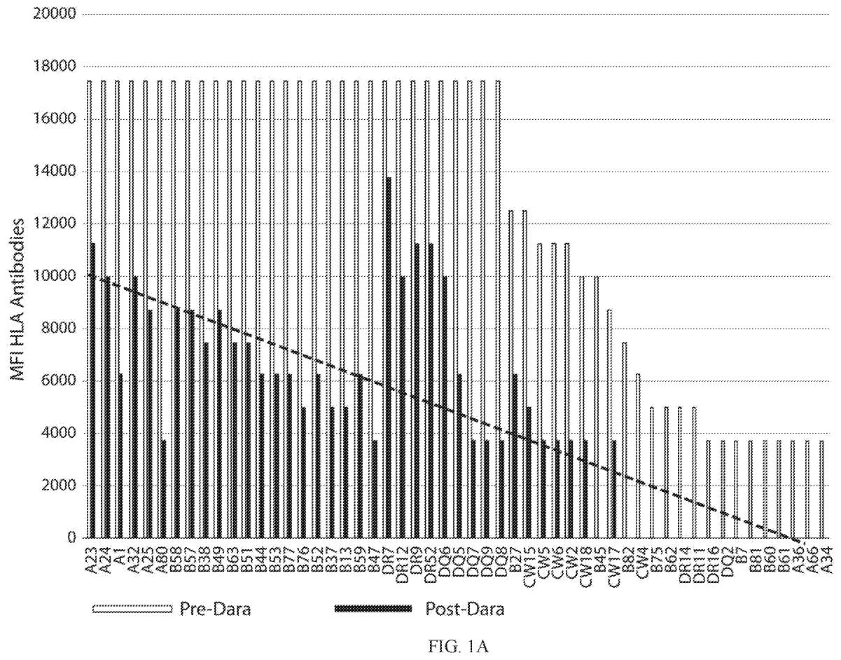

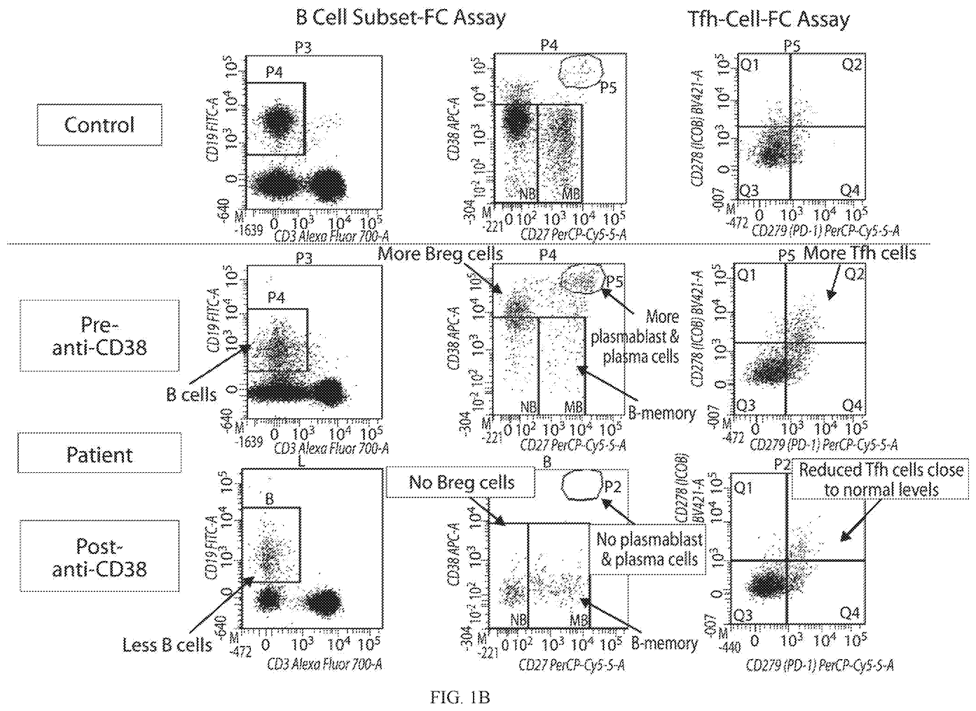

FIG. 1A shows the impact of daratumumab on HLA antibody specificities measured by LUMINEX assay. FIG. 1B shows the flow cytometry analysis of B-cells, PB and PCs pre- and post-daratumumab therapy. Here, we noted elimination of PB, PCs, B-reg (CD19+, CD38+, CD24+) and B-memory (CD19+,CD38+,CD27+) cells from the peripheral circulation after daratumumab. There was also a reduction in Tfh cells in the peripheral blood post-daratumumab.

Overall organ transplantation remains the best option for patients with cardiac and renal failure. However, sensitization to HLA antigens resulting in alloantibody formation creates an often impenetrable immunologic barrier to successful transplantation. These alloantibodies increase the risk of ABMR and reduce graft survival substantially. Current methods to reduce these alloantibodies are only modestly successful. Rituximab is often used as desensitization by targeting the CD20 protein found on B-lymphocytes. However, plasma cells (PCs) do not express CD20, likely explaining the limited success of this approach. IVIg and PLEX are used but also have limited success due to antibody rebound. Results with the PC directed proteasome inhibitor, bortezomib have also been disappointing for desensitization and ABMR treatment and was associated with significant toxicity. Despite attempts to develop tolerable therapeutics for desensitization and ABMR, none, to date, have been universally accepted or evolved to FDA approval. Lack of approved therapeutics often results in patients succumbing to end stage organ failure while awaiting transplantation or having a much shorter graft survival due to ABMR. Here, we report on the use of daratumumab which targets CD38 expressed on PCs and other immune cells.

The first in class CD38-targeting antibody, daratumumab, is currently FDA approved for the treatment of relapsed and refractory multiple myeloma (MM). Daratumumab is well tolerated with a favorable side effect profile in most studies and was in our patients as well. Daratumumab and evolving CD38-targeting antibodies eliminate CD38+ cells through classic Fc-dependent effector mechanisms, including antibody-mediated cytotoxicity (ADCC), antibody-dependent cellular phagocytosis, and complement dependent cytotoxicity (CDC). In addition, there is increasing evidence that daratumumab eliminates CD38+ immune suppressor cells, including T-regs, B-regs, and myeloid-derived suppressor cells. Daratumumab treatment results in a marked increase in T-effector cell numbers and activity. This observation may explain the divergent results obtained in our ABMR patient where resolution of ABMR was seen, but CMR intensified. We also saw evidence of increasing numbers of CD4+ T-cells post-daratumumab treatment along with elimination of B-regs. Thus, caution is required in using this agent until these issues are better clarified in highly-HLA sensitized patients.

Although daratumumab appeared to fail at reducing HLA class II antibodies including DSA to DQ5 despite reductions in HLA class I, the ABMR features were resolved post-daratumumab despite a failure to reduce DSA DQ5 antibody levels. This observation may be explained by the depletion of NK cells by daratumumab. Here, despite daratumumab's lack of effect on the DSA, the effector functions of DSAs mediating ADCC could be inhibited by NK cell depletion, limiting tissue injury to the graft.

In summary, daratumumab was well tolerated with minimal adverse events (AEs) noted. The treatment regimen showed significant reductions in circulating HLA class I & class II antibodies for the desensitization patient with elimination of circulating B-regs, B-memory, PCs and PBs. The ABMR patient was more instructive in that reductions in HLA class I persisted for 6M post-daratumumab. However, there was no meaningful impact on HLA class II antibodies, including the DSA DQ5. Class II antibodies also rebounded post-daratumumab treatment. It is possible the elimination of CD38+ B-regs and T-regs may have incited CMR progression in this patient. For this patient there is significant improvement in ABMR features despite a lack of impact on the DSA DQ5. Here, it is possible that complete elimination of ABMR features were likely aided by daratumumab's ability to deplete CD38+ natural killer (NK) cells, thus restricting ADCC. This observation correlated to an increased risk of infectious complications in daratumumab treated patients with multiple myeloma. We have also observed a significant impact of anti-CD38 in vitro on depleting NK cells from normal individuals. Thus, anti-CD38 therapy may represent a double edged sword in ABMR treatment. First, there are important effector functions such as elimination of PB and PCs that reduce DSAs, and elimination of effector cells that mediate ADCC. However, elimination of T-regs and B-regs likely predisposes to CMR. Here, despite resolution of ABMR, CMR emerged and the net effect was no significant improvement in the patient's renal function. Daratumumab and other anti-CD38 monoclonal agents, other anti-CD38 therapies, are likely good candidates for desensitization in patients awaiting life-saving transplants and may represent an important way forward. For example, isatuximab, MOR-202, GBR-1342, AMG-424, TAK-169, MT-4019ND, STI-6129, A-145D, and EDC-8 may be used for desensitization in patients awaiting life-saving transplants.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method for reducing or removing donor specific anti-human leukocyte antigen (HLA) antibodies in a subject, or treating, inhibiting, or reducing severity of antibody-mediated rejection (ABMR) response to an organ transplant in the subject, comprising:
administering to the subject an effective amount of a composition,
wherein the composition comprises an anti-CD38 antibody, a CD38-binding fragment of an antibody, immune cells expressing a chimeric antigen receptor (CAR) that comprises at least a CD38-targeting region, a polynucleotide encoding the CAR, a vector comprising the polynucleotide, or a combination thereof.

2. (canceled)

3. The method of claim 1, wherein the composition is an anti-CD38 antibody or a CD38-binding fragment of an antibody selected from the group consisting of daratumumab, isatuximab, MOR-202, GBR-1342, AMG-424, TAK-169, MT-4019ND, STI-6129, A-145D, EDC-8, or a combination thereof.

4. The method of claim 1, wherein the subject has undergone standard-of-care treatment comprising one or more of immunoglobulin administration (IVIG), rituximab administration and plasma exchange (PLEX), and the subject's response to the standard-of-care treatment is ineffective.

5. The method of claim 3, wherein the subject is further resistant or has acquired resistance to immunosuppressive treatment with one or more of eculizumab, thymoglobulin, bortezomib, carfilzomib, basiliximab, mycophenolate mofetil, tacrolimus and corticosteroids.

6. The method of claim 1, wherein the organ is a kidney, heart, liver, lung, pancreas, or intestines.

7. (canceled)

8. (canceled)

9. The method of claim 1, wherein the anti-CD38 antibody or the CD38-binding fragment comprises heavy chain complementarity determining regions (HCDR) 1 (HCDR1), 2 (HCDR2) and 3 (HCDR3) sequences of SEQ ID NOs: 6, 7 and 8, respectively, and light chain complementarity determining regions (LCDR) 1 (LCDR1), 2 (LCDR2) and 3 (LCDR3) sequences of SEQ ID NOs: 9, 10 and 11, respectively.

10. The method of claim 1, wherein the anti-CD38 antibody comprises a variable heavy region (V_{H}) of amino acid sequence of SEQ ID No: 4 and a variable light region (V_{L}) of amino acid sequence of SEQ ID No: 5.

11. The method of claim 1, wherein the anti-CD38 antibody comprises a heavy chain of SEQ ID No: 2 and a light chain of SEQ ID No: 3.

12. (canceled)

13. The method of claim 1, wherein the anti-CD38 antibody or the CD38-binding fragment is administered intravenously at a dose of about 1-4 mg/kg/week, about 4-8 mg/kg/week, about 8-12 mg/kg/week, about 12-16 mg/kg/week, about 16-20 mg/kg/week, about 20-25 mg/kg/week, about 25-30 mg/kg/week or a combination thereof.

14. The method of claim 1, wherein the anti-CD38 antibody or the CD38-binding fragment is administered subcutaneously at a dose of about 1-4 mg/kg/week, about 4-8 mg/kg/week, about 8-12 mg/kg/week, about 12-16 mg/kg/week, about 16-20 mg/kg/week, about 20-25 mg/kg/week, about 25-30 mg/kg/week or a combination thereof.

15. The method of claim 1, wherein the anti-CD38 antibody or the antigen-binding fragment thereof is administered for at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, at least 6 months, at least 7 months, at least 8 months, at least 9 months, at least 10 months, at least 11 months, at least 12 months, at least 18 months, at least 24 months, or at least 36 months.

16. The method of claim 1, administering an effect amount of the anti-CD38 antibody or the CD38-binding fragment in combination with tacrolimus and/or mycophenolate mofetil.

17. A method of preventing, stabilizing or reducing antibody-mediated rejection (ABMR) response to an organ transplant in a subject, comprising administering to the subject a prophylactically or therapeutically effective amount of an anti-CD38 antibody or anti-CD38 antibody fragment, wherein the antibody or antibody fragment comprises a variable heavy chain polypeptide comprising heavy chain complementarity determining regions (HCDR) 1 (HCDR1), 2 (HCDR2) and 3 (HCDR3) sequences of SEQ ID NOs: 6, 7 and 8, respectively, and a variable light chain polypeptide comprising light chain complementarity determining regions (LCDR) 1 (LCDR1), 2 (LCDR2) and 3 (LCDR3) sequences of SEQ ID NOs: 9, 10 and 11, respectively.

18. The method of claim 17, wherein the anti-CD38 antibody is daratumumab.

19. The method of claim 17, further comprising selecting a subject exhibiting a symptom of ABMR before or at the time of administering the anti-CD38 antibody or the anti-CD38 antibody fragment.

20. The method of claim 17, wherein the organ comprises a kidney and the symptom of ABMR is one or more of: (i) deterioration of allograft function measured by serum creatinine and estimated glomerular filtration rate (eGFR); (ii) presence of donor specific antibodies; and/or (iii) biopsy evidence of capillaritis, inflammation and complement (C4d) deposition.

21. A method for desensitizing a subject by reducing and/or eliminating donor specific anti-human leukocyte antigen (HLA) antibodies in the subject, comprising administering an effective amount of an anti-CD38 antibody or anti-CD38 antibody fragment, wherein the antibody or antibody fragment comprises a variable heavy chain polypeptide comprising heavy chain complementarity determining regions (HCDR) 1 (HCDR1), 2 (HCDR2) and 3 (HCDR3) sequences of SEQ ID NOs: 6, 7 and 8, respectively, and a variable light chain polypeptide light chain comprising complementarity determining regions (LCDR) 1 (LCDR1), 2 (LCDR2) and 3 (LCDR3) sequences of SEQ ID NOs: 9, 10 and 11, respectively.

22. The method of claim 21, wherein the anti-CD38 antibody is daratumumab.

23. The method of claim 21, wherein the anti-CD38 antibody is administered before or at the time of an organ transplantation.

24. The method of claim 21, wherein the anti-CD38 antibody is administered after an organ transplantation.
